(19) United States
(12) Patent Application Publication
(10) Pub. No.: US 2025/0346634 A1
(43) Pub. Date: Nov. 13, 2025

(54) CYCLIC PEPTIDES FOR TARGETING EPIDERMAL GROWTH FACTOR RECEPTOR AND MUTATIONS THEREOF FOR DRUG DELIVERY

(71) Applicant: Ariel Scientific Innovations Ltd., Ariel (IL)

(72) Inventors: Bat Chen Revital LUBIN, Ariel (IL); Gary GELLERMAN, Ariel (IL); Olga FURMAN, Ariel (IL); Andrii BAZYLEVICH, Ariel (IL)

(73) Assignee: Ariel Scientific Innovations Ltd., Ariel (IL)

(21) Appl. No.: 18/868,749

(22) PCT Filed: May 23, 2023
(86) PCT No.: PCT/IL2023/050533

§ 371 Date: Nov. 24, 2024

Related U.S. Application Data
(60) Provisional application No. 63/344,657, filed on May 23, 2022.

(51) Int. Cl. C07K 7/64; A61K 47/64; A61K 49/00; G01N 33/574

(52) U.S. Cl. CPC C07K 7/64; A61K 47/64; A61K 49/0056; G01N 33/57492; G01N 2333/71; G01N 2410/00

(57) ABSTRACT

Provided herein is a family of cyclic peptides that exhibit specific affinity to EGFR expressing cells, and that can be used as drug delivery vehicle for labeling agents and cytotoxic drugs against said cells in the context of a peptide-drug conjugates, as well as pharmaceutical compositions including the same, and uses thereof for treating cancer.

### RELATED APPLICATIONS

This application claims the benefit of priority of U.S. Provisional Patent Application No. 63/344,657, filed on 23 May 2022, the contents of which are incorporated herein by reference in their entirety.

### SEQUENCE LISTING STATEMENT

The file entitled 96504.xml, created on 23 May 2023, comprising 20,480 bytes, submitted concurrently with the filing of this application is incorporated herein by reference.

### FIELD AND BACKGROUND OF THE INVENTION

The present invention, in some embodiments thereof, relates to cancer treatment, and more particularly, but not exclusively, to a family of cyclic peptides that have affinity to epidermal growth factor receptor and mutations thereof for drug delivery.

The genetic and cellular heterogeneity of almost all types of cancer continues to confound the development of effective cancer chemotherapies. To solve this issue, several studies have highlighted the potential of combined treatment with drugs differing in their mechanism of action. The rationale for this strategy is that such combinations will significantly reduce the probability of the outgrowth of clones resistant to several drugs. However, such resistance does occur. One example is the use of small molecules and antibodies to treat cancers over expressing the epidermal growth factor receptor (EGFR).

EGFR plays a critical role in regulation of cell proliferation, differentiation, and migration. Its overexpression is frequently found in a variety of human tumors of epithelial origin, including non-small cell lung cancer (NSCLC), breast, head and neck, gastric, colorectal, prostate, esophageal, bladder, renal, pancreatic, and ovarian cancers. EGRF undergoes several mutations, particularly within exons 18-21, which encode a portion of the EGFR kinase domain that has been associated with some forms of lung cancers. The most common variant is EGFR variant III (EGFRvIII) which entails a deletion of exons 2-7. The EGFRvIII mutation was suggested as a marker for a cancer stem cell or tumor-initiating population and its presence is associated with a more aggressive disease and poorer prognosis. EGFR overexpression in NSCLC has been identified in between 40% to 89% of cases.

NSCLC is the most common form of lung cancer and carries with high morbidity and mortality with a 5-year overall survival rate of only 11%-15%. Drug resistance is a major cause for therapeutic failure in this disease. Two classes of EGFR inhibitors have been clinically approved, namely monoclonal antibodies (cetuximab, panitumumab), which target the extracellular domain of EGFR, and small molecule kinase inhibitors (gefitinib, erlotinib), which block the intracellular phosphorylation of the receptor. Despite a good response rate to these drugs, most patients eventually develop drug resistance—a major limitation that reduces the long-term efficacy of the therapy. Hence, new treatment approaches are still needed.

A peptide-drug conjugate (PDC) is a targeted therapeutic approach for cancer therapy that combines the specificity of peptides with the potency of small molecule drugs. In a PDC, a peptide that specifically targets cancer cells is chemically linked to a cytotoxic drug. The peptide guides the drug to the tumor site, where it is released to exert its therapeutic effect. This approach increases the selectivity and efficacy of cancer treatment while reducing side effects on healthy tissues. In the clinical trials of PDCs, two therapeutic PDCs are currently approved on the market: 177 Lu-dotatate (lutathera) and melflufen. The first PDC approved by the US Food and Drug Administration (FDA), 177 Lu-dotatate, is used to treat gastrointestinal, pancreatic, and neuroendocrine tumors. Others are in various stages of research and development.

International Patent Application No. WO2015187540A1 provides peptide-drug conjugates comprising p-aminobenzyl carbamoyl or p-aminobenzolyl carbonate self-immolating linkers. The peptide-drug conjugates comprise a peptide moiety that can be cleaved by cellular proteases, bound to the self-immolating linker, which linker is bound to a cytotoxic drug moiety. Upon cleavage of the peptide moiety, the linker self-immolates, releasing the cytotoxic drug in active form. Dimeric structures of the peptide drug conjugates comprising two molecules of cytotoxic drug per conjugate are also disclosed.

WO2022155172A1 provides conjugates of therapeutic molecules (e.g., cytotoxic agents) and targeting moieties (e.g., peptides) which are useful in the treatment of diseases such as cancer.

### SUMMARY OF THE INVENTION

The present disclosure provides a methodology for identifying and validating novel cyclic peptides that exhibit high specific affinity to EGFR and/or an EGFR mutant expressed in a cancer cells, as well as the capacity to be internalized into the cells and thereby be used as a delivery vehicle as a conjugate carrying a payload such as a drug molecule, a detectible labeling/reporter agent, and the likes.

Thus, according to an aspect of some embodiments of the present invention, there is provided a cyclic peptide that includes 7-11 amino acid residues having two terminal cysteine residues, looped via a disulfide bond between the terminal cysteine residues, and characterized by at least one of the following properties:

having an affinity to an EGFR and/or an EGFR mutant expressed in a cancer cell line selected from the group consisting of H1299, H1975 and DKMG, and a capacity for internalization by the cancer cell line as determined by flow cytometry assay using the cyclic peptide conjugated to a labeling moiety; and
specificity to the EGFR and/or the EGFR mutant as determined by competitive binding and/or internalization assay against a natural ligand of the EGFR and/or the EGFR mutant in the cancer cell line.

In some embodiments, the cyclic peptide is a 9-mer peptide, having a 7-mer sequence flanked by two terminal cysteine residues.

In some embodiments, the cyclic peptide is having a sequence selected individually from the group consisting of:

(SEQ ID No. 1)
CLRWRFGRC;
(SEQ ID No. 2)
CSAETVESC;
(SEQ ID No. 3)
CVRWRFGRC;
(SEQ ID No. 4)
CLAVEVRPC;
(SEQ ID No. 5)
CPNDSYHQC;
(SEQ ID No. 6)
CHVPGSYIC;
(SEQ ID No. 7)
CWHSLSLAC;
(SEQ ID No. 8)
CSALWASHC;
(SEQ ID No. 9)
CVNAMQSYC;
(SEQ ID No. 10)
CNWLSRTEC;
and
(SEQ ID No. 11)
CAQYTPGRC,

and any C-terminus amide, salt, hydrate or solvate thereof.

In some embodiments, the cyclic peptide is CLRWRFGRC (SEQ ID No. 1), and any C-terminus amide, salt, hydrate or solvate thereof.

In some embodiments, the cyclic peptide is CSAETVESC (SEQ ID No. 2), and any C-terminus amide, salt, hydrate or solvate thereof.

In some embodiments, the cyclic peptide is CVRWRFGRC (SEQ ID No. 3), and any C-terminus amide, salt, hydrate or solvate thereof.

In some embodiments, the cyclic peptide is CLAVEVRPC (SEQ ID No. 4), and any C-terminus amide, salt, hydrate or solvate thereof.

In some embodiments, the cyclic peptide is CPNDSYHQC (SEQ ID No. 5), and any C-terminus amide, salt, hydrate or solvate thereof.

In some embodiments, the cyclic peptide is CHVPGSYIC (SEQ ID No. 6), and any C-terminus amide, salt, hydrate or solvate thereof.

In some embodiments, the cyclic peptide is CWHSLSLAC (SEQ ID No. 7), and any C-terminus amide, salt, hydrate or solvate thereof.

In some embodiments, the cyclic peptide is CSALWASHC (SEQ ID No. 8), and any C-terminus amide, salt, hydrate or solvate thereof.

In some embodiments, the cyclic peptide is CVNAMQSYC (SEQ ID No. 9), and any C-terminus amide, salt, hydrate or solvate thereof.

In some embodiments, the cyclic peptide is CNWLSRTEC (SEQ ID No. 10), and any C-terminus amide, salt, hydrate or solvate thereof.

In some embodiments, the cyclic peptide is CAQYTPGRC (SEQ ID No. 11), and any C-terminus amide, salt, hydrate or solvate thereof.

In some embodiments, the cyclic peptide is attached to a moiety of a bioactive agent (a conjugate).

In some embodiments, the bioactive agent is selected from the group consisting of a drug, cytotoxic agent, an imaging agent, a diagnostic agent and a labeling agent.

In some embodiments, the cyclic peptide provided herein is for use in targeted drug delivery to cells overexpressing the EGFR and EGFRvIII mutation.

According to another aspect of some embodiments of the present invention, there is provided a conjugate that includes a moiety of the cyclic peptide provided herein, and a moiety of a bioactive agent. In some embodiments, the two moieties are linked covalently.

In some embodiments, the moiety of the cyclic peptide and the moiety of the bioactive agent are connected via a linking moiety.

In some embodiments, the linking moiety is labile, biocleavable, or biodegradable.

In some embodiments, the linking moiety includes a spacer moiety.

In some embodiments, the linking moiety is selected from the group consisting of a γ-aminobutyric acid (GABA) moiety, a glutathione moiety, a lysine moiety, a succinic acid moiety, a 2-amino-5-(carbamoylamino) pentanoic acid (PABA) moiety, a citrulline moiety, a valine-citrulline-PABA moiety, and any combination thereof.

In some embodiments, the bioactive agent is a cytotoxic agent.

In some embodiments, the cytotoxic agent is selected from the group consisting of camptothecin (CTP), doxorubicin (DOX), monomethyl auristatin F (MMFA), and 7-ethyl-10-hydroxycamptothecin (SN38).

In some embodiments, the peptide-drug conjugate exhibits higher cytotoxicity towards cells overexpressing the EGFR and EGFRvIII mutation than the free cytotoxic agent.

According to another aspect of some embodiments of the present invention, there is provided a pharmaceutical composition that includes the conjugate as provided herein, and a pharmaceutically acceptable carrier, diluent, or excipient.

In some embodiments, the pharmaceutical composition is packaged in a packaging material and identified in print, in or on the packaging material, for use in the treatment of a medical condition. In some embodiments, the medical condition is treatable by the bioactive agent (the drug). In some embodiments, the medical condition is cancer.

According to another aspect of some embodiments of the present invention, there is provided a peptide-drug conjugate as provided herein, for use in the treatment of a medical condition. In some embodiments, the medical condition is treatable by the bioactive agent (the drug). In some embodiments, the medical condition is cancer.

According to another aspect of some embodiments of the present invention, there is provided a peptide-drug conjugate as provided herein, for use in the preparation of a medicament.

According to another aspect of some embodiments of the present invention, there is provided a use of the peptide-drug conjugate as provided herein, in the preparation of a medicament.

In some embodiments, the medicament is for treating a medical condition. In some embodiments, the medical condition is treatable by the bioactive agent. In some embodiments, the medical condition is cancer.

According to another aspect of some embodiments of the present invention, there is provided a use of the conjugate provided herein for treating a medical condition. In some embodiments, the medical condition is treatable by the bioactive agent. In some embodiments, the medical condition is cancer.

According to another aspect of some embodiments of the present invention, there is provided a method for treating a medical condition associated with cells overexpressing the EGFR and EGFRvIII mutation, that includes administering to a subject in need thereof a therapeutically effective amount of the conjugate or the pharmaceutical composition or the medicament, as provided herein.

In some embodiments, the medical condition is treatable by the bioactive agent.

In some embodiments, the medical condition is cancer.

In some embodiments, the medical condition is associated with cells overexpressing the EGFR and EGFRvIII mutation.

In some embodiments, the bioactive agent is a cytotoxic agent, or an anticancer drug.

According to another aspect of some embodiments of the present invention, there is provided a method for diagnosing a disease associated with cells overexpressing the EGFR and EGFRvIII mutation, that includes using the conjugate provided herein as a diagnostic agent in imaging or detection techniques. In some embodiments, the bioactive agent is a detectible labeling moiety.

As used herein the term “about” refers to +10%. For example, the term “about 100 μm” encompasses the value 100 μm, as well as the values 90 μm, 91 μm, 92 μm, 93 μm, 94 μm, 95 μm, 96 μm, 97 μm, 98 μm, 98 μm, 99 μm, 100 μm, 101 μm, 102 μm, 103 μm, 104 μm, 105 μm, 106 μm, 107 μm, 108 μm, 109 μm, and 110 μm.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrase “selected from the group consisting of” includes all members of the recited group, each member of the recited group, and all possible combinations. For example, selected from the group consisting of A, B, and C, includes A, only, as well as B, only, as well as C, only, as well as A and B, as well as A and C, as well as B and C, and as well as A, B, and C.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

When applied to an original property, or a desired property, or an afforded property of an object or a composition, the term “substantially maintaining”, as used herein, means that the property has not change by more than 20%, 10% or more than 5% in the processed object or composition.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### DESCRIPTION OF EMBODIMENTS OF THE INVENTION

The present invention, in some embodiments thereof, relates to cancer treatment, and more particularly, but not exclusively, to a family of cyclic peptides that have affinity to epidermal growth factor receptor and mutations thereof for drug delivery.

Before explaining at least one embodiment of the invention in detail, it is to be understood that the invention is not necessarily limited in its application to the details set forth in the following description or exemplified by the Examples. The disclosure is meant to encompass other embodiments or of being practiced or carried out in various ways.

While conceiving the present invention, the present inventors contemplated a strategy that could effectively overcome the hurdle of drug resistance is to employ drug delivery systems that target overexpressed survival and proliferation-related receptors, to specifically deliver cytotoxic drugs into cancer cells. In this scenario, the use of S—S bridged cyclic peptides incorporated into Peptide-Drug conjugates (PDCs) carries a number of key advantages. PDCs are conformationally more constraint structures with enhanced metabolic stability and binding affinity/specificity to target molecules compared to their linear counterparts, which enhances pharmacokinetics and effectiveness of the drug delivery to cancer cells.

While reducing the present invention to practice, the present inventors have discovered and demonstrated a family of cyclic peptide ligands that selectively bind to both EGFR and EGFRvIII and evaluate the targeting cytotoxicity of their PDCs bearing Topo I inhibitor Camptothecin (CPT) of NSCLC cell lines.

### Cyclic Peptides:

As discussed above, the EGF-EGFR pathway has become a main focus for selective chemotherapeutic intervention. As a result, two classes of EGFR inhibitors have been clinically approved, namely monoclonal antibodies (cetuximab and panitumumab), which target the extracellular domain of EGFR, and small molecule kinase inhibitors (gefitinib and erlotinib), which block the intracellular phosphorylation of the receptor. Despite a good response rate to these drugs, most patients develop drug resistance. Therefore, new treatment approaches are needed.

In the course of reducing the present invention to practice, phage display technology was used to discover the EGFR-specific cyclic peptides provided herein, that would act as drug carriers in a targeted drug delivery system. For peptide selection, the inventors utilized viable cells instead of pure proteins. The selection against the isolated biomarker is more accurate, so that irrelevant molecules do not obscure the receptor of interest, and the search against whole cells brings the experiment closer to natural environment. To mimic the more natural environment, phage display biopanning procedure was used on H1299, H1975, and DKMG cells that have overexpression of EGFR or a mutation of EGFR on the cell surface.

Furthermore, combining phage display biopanning with Next-Generation Sequencing (NGS) technology allowed the inventors to perform the high throughput screening and selection of EGFR-specific peptides. NGS is a massively parallel sequencing technology that offers ultra-high throughput, scalability, and speed. The technology is used to determine the order of nucleotides in entire genomes or targeted regions of DNA or RNA.

### General Structure:

In the context of the present invention, a cyclic peptide is a type of peptide in which the amino acid sequence forms a ring (a loop) structure rather than a straight (linear) chain. The cyclic peptides, according to some embodiments of the present invention, include 7-11 amino acid residues in their sequence, wherein each peptide sequence exhibits a terminal cysteine residue at each of the N- and C-terminus.

The peptides' termini can exhibit a free amino and/or carboxyl group, respectively, or a protected amino and/or carboxyl group. In some embodiments, the C-terminus is an amide group (C-amide).

The peptide, or conjugates thereof, can be in the form of a salt, a hydrate, or a solvate. Affinity criterion for peptide selection:

The cyclic peptides provided herein are characterized by an affinity for the epidermal growth factor receptor (EGFR) or an EGFR mutant, such as the EGFRvIII mutant. This means that the peptide can bind specifically to these receptors, which are proteins found on the surface of cells. Cells having EGFR or EGFR mutant expressed on their surface are defined herein as cells that overexpress EGFR and/or EGFRvIII mutation, and referred to herein for short as “EGFR+ve cells”. In the context of the present invention, cell lines that do not express the target receptor are referred to as “EGFR−ve cells”.

The affinity of the cyclic peptides provided herein for the EGFR or EGFR mutant is determined by a process called biopanning, using a phage display library of random cyclic peptides against cells lines that express EGFR or EGFR mutant for positive (specific) binding, and cell lines that do not express the target receptor, as a control for negative (nonspecific) binding.

As known in the art, and will be understood by any person of ordinary skills in the art, biopanning is an affinity selection technique that selects for peptides that bind to a given target. It involves several steps or cycles, including providing a phage display library, capturing the phage library to the desired target, washing away unbound phages, and eluting the bound phages. When a peptide “passes” a biopanning cycle, it means that it has successfully bound to the target protein and has not been washed away during the washing step. The bound phages are then eluted and amplified for additional rounds of biopanning to enrich for phages displaying peptides with high affinity for the target protein.

More specifically, biopanning process for determining that a given cyclic peptide exhibit specific affinity to the target receptor involves incubating a library of bacteriophages (viruses that infect bacteria) displaying a very large number (about one billion; 10⁹) of random and unique cyclic peptides on their surface with at least one EGFR+ve cell line (specific binding) and at least one EGFR−ve cell line (nonspecific binding). Nonspecific binding or weakly binding phages are then removed, and the remaining phages are amplified and subjected to additional rounds of biopanning to enrich for phages displaying peptides with high affinity for the target protein.

In an example of some embodiments of the present invention, the phage display peptide library that can be used for biopanning is the Ph.D.-C7C™ Kit (7-mer cyclic peptides) by New England Biolabs Inc., that contains the Ph.D.-C7C™ Phage Display Peptide Library, and −96gIII sequencing primer for >50 sequencing reactions. In this embodiment, the Ph.D.-C7C Phage Display Peptide Library is based on a combinatorial library of random disulfide looped peptides fused to the N-terminus of a minor coat protein (pIII) of M13 phage. The library consists of about 10⁹ electroporated (i.e., unique) sequences.

According to some embodiments of the present invention, the cyclic peptides are characterized by “passing” three cycles of biopanning, as described hereinabove. Alternatively, each of the cyclic peptides provided herein is also characterized by having an affinity to an EGFR and/or an EGFR mutant as determined by binding to the EGFR and/or an EGFR mutant in at least three cycles of biopanning using a phage display library of random cyclic peptides against at least one EGFR+ve cell line and at least one EGFR−ve cell line. This process is typically repeated for at least three cycles to ensure that the selected peptides have a high affinity for the target protein.

This method enabled the inventors to identify and synthetize a family of exemplary cyclic peptide candidates and study their EGFR specificity and selectivity.

### Internalization (Uptake) by an EGFR+ve Cell Line:

The cyclic peptides provided herein are characterized by the capacity to be internalized into an EGFR+ve cell upon binding to an EFGR or a mutant thereof. For example, the cyclic peptides exhibit binding and internalization into cancer cells, such as H1299, H1975 and DKMG cell lines. This capacity can be determined by methods known in the art. For example, internalization of the cyclic peptide can be determined by flow cytometry using a cyclic peptide labeled by a detectable moiety, or a labeling moiety, such as a fluorescent moiety. An exemplary labeling moiety, according to some embodiments of the present invention, can be fluorescein 5-isothiocyanate (FITC).

An exemplary internalization assay is provided in the Examples section that follows below.

### Specificity Towards EGFR:

A cyclic peptide provided herein is also characterized by specificity towards an EGFR, as determined by competitive binding and internalization assay against a ligand of EGFR, preferably a natural ligand of the receptor that is overexpressed in cancer cell lines. An example of a natural ligand is human epidermal growth factor (hEGF).

The binding specificity of the cyclic peptide to EGFR can be determined by comparing the level of binding and/or internalization of the peptide to the receptor in the absence and presence of a known ligand of the receptor; if the binding and/or internalization of the peptide is reduced.

A person of ordinary skills in the art would appreciate and be familiar with the tools and methodologies for assaying specificity, for example by competitive flow cytometry using a labeled cyclic peptide, EGF and at least one EGFR+ve cell line.

Exemplary competitive binding assays are provided in the Examples section that follows below.

### Additional Properties of the Cyclic Peptides:

As demonstrated in the Examples section that follows below, confocal microscopy and peptide docking was used to further confirm the fluorescence-activated cell sorting results (FACS). Cytotoxicity assay was done to study the peptide and the peptide-drug conjugate activity.

### Specific Exemplary Cyclic Peptides:

An exemplary group of 11 peptides were selected for further investigation after identification using biopanning binding, internalization assay, and specificity by competitive assay.

Thus, according to some embodiments of the present invention, there is provided a family of cyclic peptides, as listed in Table 1 below, presenting the sequence as a cyclic (“c”) peptide closed by disulfide bond between the sulfur atoms of the cysteine residues:

TABLE 1

| | | | SEQ |
|---|---|---|---|
| Peptide | | | ID |
| denotation | Sequence | Structure | No. |
| P1 | c(CLRWRFGRC) | 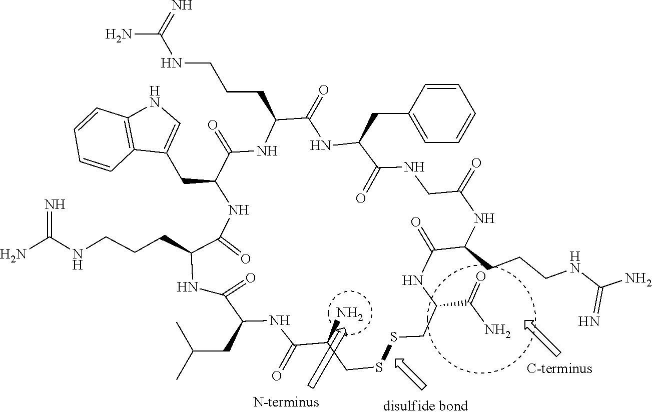 | 1 |
| P2 | c(CSAETVESC) | 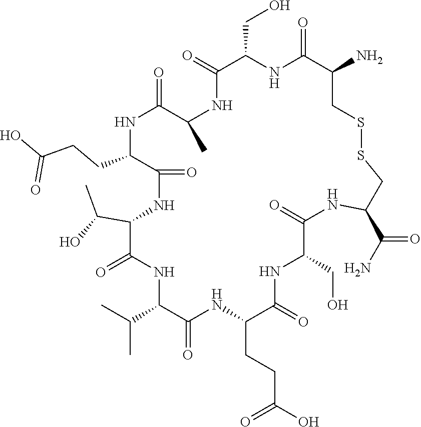 [SMILES: CC(C)[C@@H]1NC(=O)[C@H]([C@@H](C)O)NC(=O)[C@H](CCC(=O)O)NC(=O)[C@H](C)NC(=O)[C@H](CO)NC(=O)[C@@H](N)CSSC[C@@H](C(N)=O)NC(=O)[C@H](CO)NC(=O)[C@H](CCC(=O)O)NC1=O] | 2 |
| P3 | c(CVRWRFGRC) | 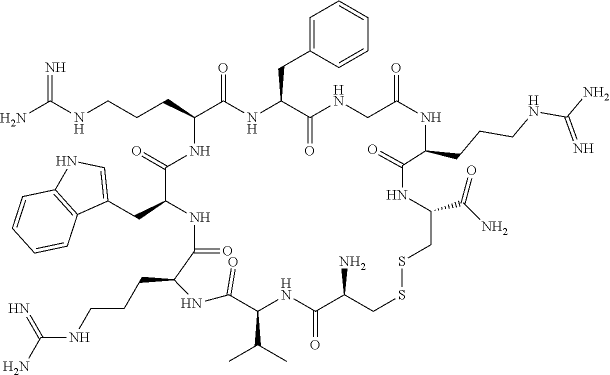 [SMILES: CC(C)[C@@H]1NC(=O)[C@@H](N)CSSC[C@@H](C(N)=O)NC(=O)[C@H](CCCNC(=N)N)NC(=O)CNC(=O)[C@H](Cc2ccccc2)NC(=O)[C@H](CCCNC(=N)N)NC(=O)[C@H](Cc2c[nH]c3ccccc23)NC(=O)[C@H](CCCNC(=N)N)NC1=O] | 3 |
| P4 | c(CLAVEVRPC) | 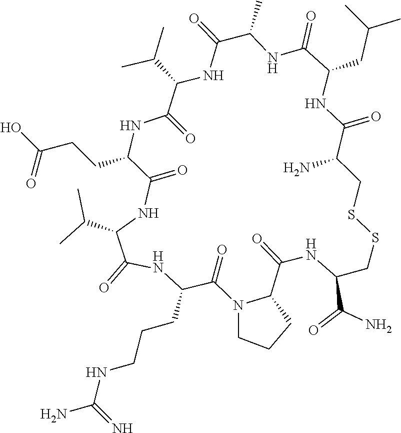 [SMILES: CC(C)C[C@@H]1NC(=O)[C@@H](N)CSSC[C@@H](C(N)=O)NC(=O)[C@@H]2CCCN2C(=O)[C@H](CCCNC(=N)N)NC(=O)[C@H](C(C)C)NC(=O)[C@H](CCC(=O)O)NC(=O)[C@H](C(C)C)NC(=O)[C@H](C)NC1=O] | 4 |
| P5 | c(CPNDSYHQC) | 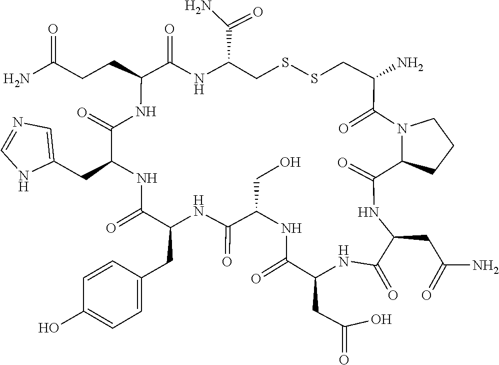 [SMILES: NC(=O)CC[C@@H]1NC(=O)[C@H](Cc2cnc[nH]2)NC(=O)[C@H](Cc2ccc(O)cc2)NC(=O)[C@H](CO)NC(=O)[C@H](CC(=O)O)NC(=O)[C@H](CC(N)=O)NC(=O)[C@@H]2CCCN2C(=O)[C@@H](N)CSSC[C@@H](C(N)=O)NC1=O] | 5 |
| P6 | c(CHVPGSYIC) | 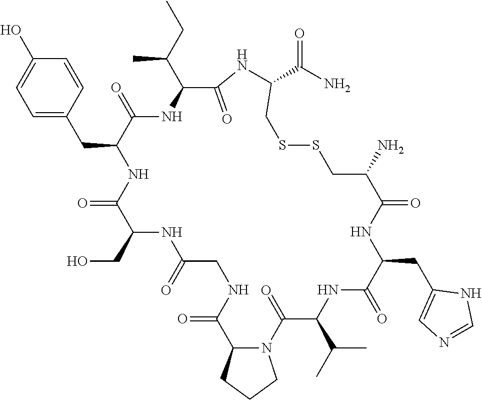 [SMILES: CC[C@H](C)[C@@H]1NC(=O)[C@H](Cc2ccc(O)cc2)NC(=O)[C@H](CO)NC(=O)CNC(=O)[C@@H]2CCCN2C(=O)[C@H](C(C)C)NC(=O)[C@H](Cc2cnc[nH]2)NC(=O)[C@@H](N)CSSC[C@@H](C(N)=O)NC1=O] | 6 |
| P7 | c(CWHSLSLAC) | 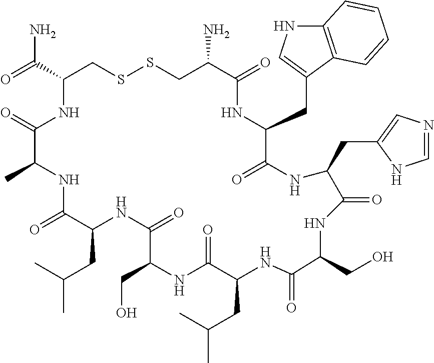 [SMILES: CC(C)C[C@@H]1NC(=O)[C@H](CO)NC(=O)[C@H](CC(C)C)NC(=O)[C@H](CO)NC(=O)[C@H](Cc2cnc[nH]2)NC(=O)[C@H](Cc2c[nH]c3ccccc23)NC(=O)[C@@H](N)CSSC[C@@H](C(N)=O)NC(=O)[C@H](C)NC1=O] | 7 |
| P8 | c(CSALWASHC) | 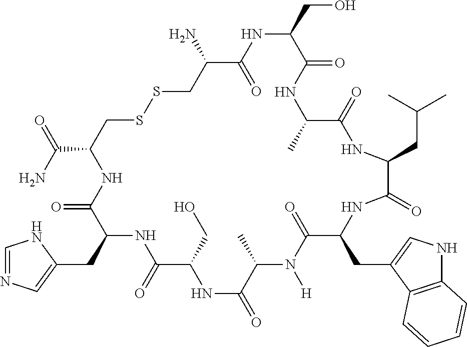 [SMILES: CC(C)C[C@@H]1NC(=O)[C@H](C)NC(=O)[C@H](CO)NC(=O)[C@@H](N)CSSC[C@@H](C(N)=O)NC(=O)[C@H](Cc2cnc[nH]2)NC(=O)[C@H](CO)NC(=O)[C@H](C)NC(=O)[C@H](Cc2c[nH]c3ccccc23)NC1=O] | 8 |
| P9 | c(CVNAMQSYC) | 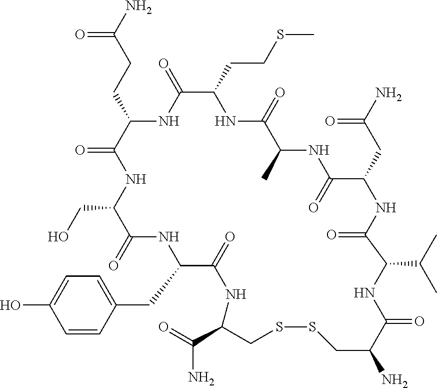 [SMILES: CSCC[C@@H]1NC(=O)[C@H](C)NC(=O)[C@H](CC(N)=O)NC(=O)[C@H](C(C)C)NC(=O)[C@@H](N)CSSC[C@@H](C(N)=O)NC(=O)[C@H](Cc2ccc(O)cc2)NC(=O)[C@H](CO)NC(=O)[C@H](CCC(N)=O)NC1=O] | 9 |
| P10 | c(CNWLSRTEC) | 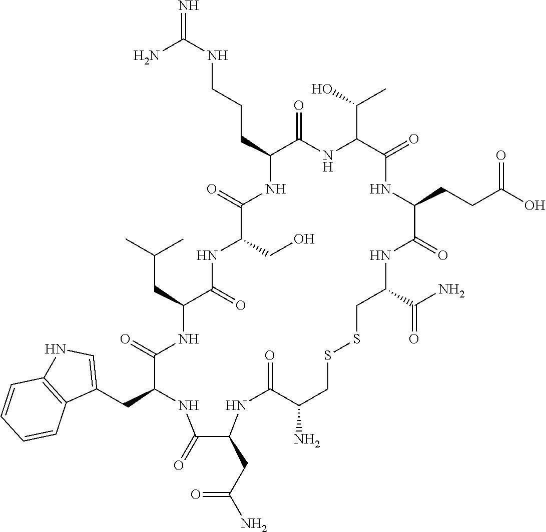 [SMILES: CC(C)C[C@@H]1NC(=O)[C@H](Cc2c[nH]c3ccccc23)NC(=O)[C@H](CC(N)=O)NC(=O)[C@@H](N)CSSC[C@@H](C(N)=O)NC(=O)[C@H](CCC(=O)O)NC(=O)C([C@@H](C)O)NC(=O)[C@H](CCCNC(=N)N)NC(=O)[C@H](CO)NC1=O] | 10 |
| P11 | c(CAQYTPGRC) | 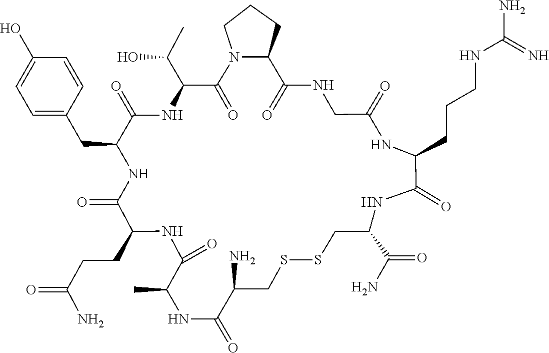 [SMILES: C[C@@H]1NC(=O)[C@@H](N)CSSC[C@@H](C(N)=O)NC(=O)[C@H](CCCNC(=N)N)NC(=O)CNC(=O)[C@@H]2CCCN2C(=O)[C@H]([C@@H](C)O)NC(=O)[C@H](Cc2ccc(O)cc2)NC(=O)[C@H](CCC(N)=O)NC1=O] | 11 |

Although Table 1 presents the structures of the cyclic peptides in the form of an C-terminus amide, the C-terminal of each of the peptides can be in the form of a carboxyl group or an amide group.

In some embodiments, since it is cyclized via the side-chain thiol of the terminal cysteines, the cyclic peptide can be in the form of a free N-terminal amine, a free C-terminal carboxyl, a terminal amide, a terminal ester, a salt, preferably a pharmaceutically acceptable salt, a hydrate thereof or a solvate thereof.

The term “solvate” refers to a complex of variable stoichiometry (e.g., di-, tri-, tetra-, penta-, hexa-, and so on), which is formed by a solute (the compound as described herein) and a solvent, whereby the solvent does not interfere with the biological activity of the solute. Suitable solvents include, for example, ethanol, acetic acid and the like. The term “hydrate” refers to a solvate, as defined hereinabove, where the solvent is water.

In some embodiments, the cyclic peptide comprises only residues of naturally occurring amino acids. In some embodiments, the cyclic peptide comprises at least one residue of a D-amino acid that is not glycine.

The cyclic peptides have shown specific and selective binding and internalization to the EGFR+ve cells. Off target cells study demonstrated that the two cyclic peptides denoted P6 and P9 exhibit high specific activity to the EGFR. Confocal microscopy demonstrated high specificity of P6 to the NSCLC while P9 was more specific to the glioblastoma cells. XTT assay on EGFR overexpressed cell lines have shown that the conjugates of P6 and P9 with Camptothecin (CPT) have higher cytotoxicity compared to the free CPT. The results led to the discovery of the instantly disclosed cyclic peptides specifically targeted the EGF receptor and its specific mutation EGFRvIII.

### Peptide-Drug Conjugate:

According to an aspect of some embodiments of the present invention, there is provided a family of EGFR-specific short cyclic peptides that can be used for targeted drug delivery in the context of a peptide-drug conjugate (PDC).

The ability of the peptide-drug conjugates to remain stable and also to specifically enter the target cells is an important factor in the delivery of highly toxic drugs, and indeed, the cyclic peptides selected as candidates for targeted cell binding had the ability to be taken up by the cells, showing high internalization into DKMG cells carrying the EGFRvIII mutation, a genetic marker of several cancers. EGFRvIII mutation has also suggested as a marker for a cancer stem cell or tumor-initiating population, as this mutation is associated with a more aggressive disease and poorer prognosis.

As presented hereinbelow, competitive binding assay was used to determine the ability of labeled peptides to compete with the natural ligand of the EGF receptor. FACS analysis has showed that EGF successfully competed with the cyclic peptide provided herewith, thus the number of FITC-positive cells, with bound peptides after the incubation was reduced significantly. After the co-incubation with natural ligand EGF, it was found that binding activity was significantly reduced. This result proved the interplay on the EGFR receptors suggesting their probable role in the cyclic peptides delivery system transport. Further investigation of the herein-provided cyclic peptides for their target specificity revealed that some showed high specificity to the EGFR expressing cells.

The Examples section that follows also present peptide docking conducted for the cyclic peptides provided herein, which confirmed the target specificity and showed binding at the active site of the receptor. The cyclic peptides exhibited good binding energy that ranged from −13.0 to −11.3 kcal mol⁻¹.

Since EGF stimulate cell proliferation, the safety of the cyclic peptides provided herein needed to be tested in a risk of stimulating cell proliferation. The results presented below showed that by themselves, the peptides did not stimulate cell proliferation and therefore prove worthy for safe use as targeting ligands and drug carriers in the context of a peptide-drug conjugate. This was further confirmed by observing a low cytotoxic effect of the free peptides compared to the more pronounced cytotoxicity when the peptides were conjugated to CPT.

Thus, according to an aspect of some embodiments of the present invention, there is provided a peptide-drug conjugate, that includes a cyclic peptide, according to embodiments of the present invention, and at least one bioactive agent, e.g., a drug, wherein the moieties of the cyclic peptide and the bioactive agent are covalently connected to one-another via a linking moiety, denoted “L” in the illustrative scheme presented below.

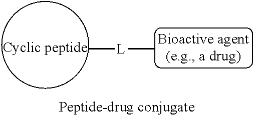

According to aspects of some embodiments of the present invention, the bioactive agent, or moiety thereof, refer to a compound (molecule) that is attached to the cyclic peptide with the intention to be delivered to a target and exert an effect under physiological conditions. In the context of the present embodiments, the terms “bioactive agent”, “pharmaceutically active agent” and “drug” are used interchangeably, however, a bioactive agent may be a molecule used for diagnostic purposes, and/or as a labeling agent for imaging, radiotherapy, and research.

As used herein, the terms “bioactive agent” and “drug” refer to small molecules or biomolecules that alter, inhibit, activate, or otherwise affect a biological mechanism or event. Bioactive agent that can be tethered to the cyclic peptide, according to embodiments of the present invention, include, without limitation, anticancer substances for all types and stages of cancer and cancer treatments (chemotherapeutic, proliferative, acute, genetic, spontaneous etc.), anti-proliferative agents, chemosensitizing agents, anti-inflammatory agents (including steroidal and non-steroidal anti-inflammatory agents and anti-pyretic agents), antimicrobial agents (including antibiotics, antiviral, antifungal, anti-parasite, anti-protozoan etc.), anti-oxidants, hormones, anti-hypertensive agents, anti-AIDS substances, anti-diabetic substances, immunosuppressants, enzyme inhibitors, neurotoxins, opioids, hypnotics, anti-histamines, lubricants, tranquilizers, anti-convulsants, muscle relaxants and anti-Parkinson substances, antipruritic agents, anti-spasmodics and muscle contractants including channel blockers, miotics and anti-cholinergics, anti-glaucoma compounds, modulators of cell-extracellular matrix interactions including cell growth inhibitors and anti-adhesion molecules, vitamins, vasodilating agents, inhibitors of DNA, RNA or protein synthesis, analgesics, anti-angiogenic factors, anti-secretory factors, anticoagulants and/or anti-thrombotic agents, anesthetics, ophthalmics, prostaglandins, anti-depressants, anti-psychotic substances, anti-emetics, radioactive agents and imaging agents. A more comprehensive listing of exemplary drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, edited by Susan Budavari et al., CRC Press, 1996, and the United States Pharmacopeia-25/National Formulary-20, published by the United States Pharmcopeial Convention, Inc., Rockville Md., 2001.

As used herein, the term “small molecule” refers to molecules, whether naturally-occurring or artificially created (e.g., via chemical synthesis), that have a relatively low molecular weight. Typically, small molecules are monomeric and have a molecular weight of less than about 1500 Da. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§ 330.5, 331 through 361, and 440 through 460; drugs for veterinary use listed by the FDA under 21 C.F.R. §§ 500 through 589, are all considered acceptable for use in accordance with the present invention.

### Anticancer Drugs:

Of particular interest for conjugation to the cyclic peptides provided herein, due to their affinity to EGFR expressing cells, are anticancer drugs. Anticancer drugs, which are contemplated as the bioactive agent in the context of a peptide-drug conjugate. according to embodiments of the present invention, include, without limitation, Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Camptothecin (CPT); Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

Approved chemotherapy agents, which are contemplated as the bioactive agent in the context of a peptide-drug conjugate. according to embodiments of the present invention, include, without limitation, abarelix, aldesleukin, aldesleukin, alemtuzumab, alitretinoin, allopurinol, altretamine, amifostine, anastrozole, arsenic trioxide, asparaginase, azacitidine, bevacuzimab, bexarotene, bleomycin, bortezomib, busulfan, calusterone, capecitabine, carboplatin, carmustine, celecoxib, cetuximab, cisplatin, cladribine, clofarabine, cyclophosphamide, cytarabine, dacarbazine, dactinomycin, actinomycin D, Darbepoetin alfa, Darbepoetin alfa, daunorubicin liposomal, daunorubicin, decitabine, Denileukin diftitox, dexrazoxane, dexrazoxane, docetaxel, doxorubicin, dromostanolone propionate, Elliott's B Solution, epirubicin, Epoetin alfa, erlotinib, estramustine, etoposide, exemestane, Filgrastim, floxuridine, fludarabine, fluorouracil 5-FU, fulvestrant, gefitinib, gemcitabine, gemtuzumab ozogamicin, goserelin acetate, histrelin acetate, hydroxyurea, Ibritumomab Tiuxetan, idarubicin, ifosfamide, imatinib mesylate, interferon alfa 2a, Interferon alfa-2b, irinotecan, lenalidomide, letrozole, leucovorin, Leuprolide Acetate, levamisole, lomustine, CCNU, meclorethamine, nitrogen mustard, megestrol acetate, melphalan, L-PAM, mercaptopurine 6-MP, mesna, methotrexate, mitomycin C, mitotane, mitoxantrone, nandrolone phenpropionate, nelarabine, Nofetumomab, Oprelvekin, Oprelvekin, oxaliplatin, paclitaxel, palifermin, pamidronate, pegademase, pegaspargase, Pegfilgrastim, pemetrexed disodium, pentostatin, pipobroman, plicamycin mithramycin, porfimer sodium, procarbazine, quinacrine, Rasburicase, Rituximab, sargramostim, sorafenib, streptozocin, sunitinib maleate, tamoxifen, temozolomide, teniposide VM-26, testolactone, thioguanine 6-TG, thiotepa, thiotepa, topotecan, toremifene, Tositumomab, Trastuzumab, tretinoin ATRA, Uracil Mustard, valrubicin, vinblastine, vinorelbine, zoledronate and zoledronic acid.

### Linking Moiety:

In general, the linking moiety can be formed during a chemical reaction, such that by reacting two or more reactive groups, the linking moiety is formed as a new chemical entity which can comprise a bond (between two atoms), or one or more bonded atoms. Alternatively, the linking moiety can be an independent chemical moiety comprising two or more reactive groups to which the reactive groups of other compounds can be attached, either directly or indirectly, as is detailed hereinunder.

Variable “L” in the above illustrative scheme, represents a linking moiety, connecting between the moiety of the cyclic peptide and the moiety of the bioactive agent (drug; detectable/diagnostic agent). The linking moiety is a result of the conjugation reaction between the two parts of the conjugate, and can be a bond (a pair of electrons forming a covalent bond), an atom-typically a heteroatom (N, O, S, and the like), or a group of atoms.

As used herein, the words “link”, “linked”, “linkage” “linker”, “bound” or “attached”, are used interchangeably herein and refer to the presence of at least one covalent bond between species, unless specifically noted otherwise. As used herein, the term “moiety” describes portion of a molecule, and typically a major portion thereof, or a group of atoms pertaining to a specific function.

As used herein, the term “linking moiety” describes a chemical moiety (a group of atoms or a covalent bond) that links two chemical moieties via one or more covalent bonds. A linking moiety may include atoms that form a part of one or both of the chemical moieties it links, and/or include atoms that do not form a part of one or both of the chemical moieties it links. For example, a peptide bond (amide) linking moiety that links two amino acids includes at least a nitrogen atom and a hydrogen atom from one amino acid and at least a carboxyl of the other amino acid.

The positions at which the bioactive agent is linked to the cyclic peptide moiety are generally selected such that once cleaved, any remaining moiety stemming from the linking moiety on the bioactive agent, if at all, does not substantially preclude its biological activity (mechanism of biological activity). Suitable positions depend on the type of bioactive agent. According to some embodiments of the present invention, the linking moieties are form such that the biological activity of the bioactive agent, once released from the cyclic peptide moiety, is not abolished and remains substantially the same as the biological activity of a similar pristine bioactive agent.

The phrase “reactive group”, as used herein, refers to a chemical group that is capable of undergoing a chemical reaction that typically leads to the formation a covalent bond. Chemical reactions that lead to a bond formation include, for example, cycloaddition reactions (such as the Diels-Alder's reaction, the 1,3-dipolar cycloaddition Huisgen reaction, and the similar “click reaction”), condensations, nucleophilic and electrophilic addition reactions, nucleophilic and electrophilic substitutions, addition and elimination reactions, alkylation reactions, rearrangement reactions and any other known organic reactions that involve a reactive group.

Representative examples of reactive groups include, without limitation, acyl halide, aldehyde, alkoxy, alkyne, amide, amine, aryloxy, azide, aziridine, azo, carbamate, carbonyl, carboxyl, carboxylate, cyano, diene, dienophile, epoxy, guanidine, guanyl, halide, hydrazide, hydrazine, hydroxy, hydroxylamine, imino, isocyanate, nitro, phosphate, phosphonate, sulfinyl, sulfonamide, sulfonate, thioalkoxy, thioaryloxy, thiocarbamate, thiocarbonyl, thiohydroxy, thiourea and urea, as these terms are defined hereinafter.

According some embodiments of the present invention, various elements of the cyclic peptide moiety presented herein are attached to one or more linking moieties via spacer moieties. As used herein, the phrase “spacer moiety” describes a chemical moiety that typically extends between two chemical moieties and is attached to each of the chemical moieties via covalent bonds. The spacer moiety may be linear or cyclic, be branched or unbranched, rigid or flexible.

The nature of the spacer moieties can be regarded as having an effect on two aspects, the synthetic aspect, namely the influence of the spacer moieties on the process of preparing the cyclic peptide moiety, and the influence of the spacer moieties on the biology activity of the cyclic peptide moiety or the bioactive agent moiety (i.e., the drug), bioavailability and other ADME-Tox considerations.

According to some embodiments of the present invention, the spacer moieties are selected such that they allow and/or promote the conjugation reaction between the cyclic peptide moiety and the bioactive agent moiety, and reduce the probability for the formation of side-products due to undesired reactions. Such traits can be selected for in terms of spacer's length, flexibility, structure and specific chemical reactivity or lack thereof. Spacer moieties with fewer reactive groups will present a simpler synthetic challenge, requiring less protection/deprotection steps and affording higher chemical yields. For example, saturated and linear alkyls of 1-10, or 1-5 carbon atoms, having one reactive group at the end atom for conjugation with a corresponding reactive group, would afford substantially higher yield and fewer side products. Similarly, a spacer moiety based on one or two chained benzyl rings would also lead to an efficient conjugation reaction.

According to some embodiments of the present invention, the spacer moieties are selected such that they provide favorable cleavage conditions for releasing the bioactive agent from the cyclic peptide, as these are discussed herein. For example, a spacer may alter the accessibility of an enzyme to the linking moiety, thereby allowing the enzyme to cleave the linkage between the bioactive agent moiety and the cyclic peptide moiety.

According to some embodiments of the present invention, the spacer moieties include, without limitation, —CH₂—, —CH₂—O—, —(CH₂)₂—, —(CH₂)₂—O—, —(CH₂)₃—, —(CH₂)₃—O—, —(CH₂)₄—, —(CH₂)₅—, —(CH₂)₆—, —(CH(CH₃))—CH₂—, —CH═CH—CH═CH—, —C═C—C═C—, —CH₂CH(OH)CH₂—, —CH₂—O—CH₂—, —CH₂—O—CH₂—O—, —(CH₂)₂—O—(CH₂)₂—, —(CH₂)₂—O—(CH₂)₂—O—, —CH₂-mC₆H₄—CH₂—, —CH₂- mC₆H₄—CH₂—O—, —CH₂-pC₆H₄—CH₂—, —CH₂-pC₆H₄—CH₂—O—, —CH₂—NHCO—, —C₆H₄—NHCO—, —CH₂—O—CH₂— and —CH═CH—CH₂—NH—(CH₂)₂—.

In some embodiments, a spacer moiety can be regarded as part of a linking moiety.

According to some embodiments of the present invention, the linking moiety is stable at physiological conditions, namely the linking moiety does not disintegrate for the duration of exposure to the physiological environment in the bodily site. Such linking moiety is referred to herein a “biostable”. Biostable linking moieties offer the advantage of an extended period of time at which the molecular structure can exert its biological activity (releasing bioactive agents at the targeted bodily site), up to the time it is secreted or otherwise removed from the bodily site. It is noted that biostability is also used as a relative term, meaning that a biostable linking moiety takes longer to break or requires certain cleavage conditions which hare less frequently encountered by the molecular structure when present in physiological conditions.

In the context of some embodiments of the present invention, biocleavable linking moieties are selected so as to break and release the bioactive agent attached thereto at certain conditions, referred to herein as “drug-releasing conditions” or “cleavage conditions”.

Examples of linking moieties, according to some embodiments of the present invention, include without limitation, amine (N; secondary and tertiary), ether (O), thioether(S), amide, carbonate, lactone, lactam, carboxylate, carbamate, ester, boroalkyl, boronate, sulphone, sulphate, phosphonate, phosphine, phosphite, cycloalkene, cyclohexene, heteroalicyclic, heteroaryl, triazine, triazole, disulfide, imine, imide, oxime, aldimine, ketimine, hydrazone, semicarbazone, acetal, ketal, aminal, aminoacetal, thioacetal, thioketal, phosphate ester, and the like. Other linking moieties are defined hereinbelow, and further other linking moieties are contemplated within the scope of the term as used herein.

According to some embodiments, the linking moiety is selected from the group consisting of:

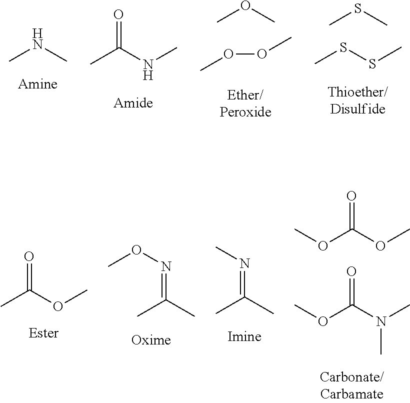

According to some embodiments of the present invention, some of the linking moieties are biocleavable-linking moieties. As used herein, the terms “biocleavable” and “biodegradable” are used interchangeably to refer to moieties that degrade (i.e., break and/or lose at least some of their covalent structure) under physiological or endosomal conditions. Biodegradable moieties are not necessarily hydrolytically degradable and may require enzymatic action to degrade.

As used herein, the terms “biocleavable moiety” or “biodegradable moiety” describe a chemical moiety, which undergoes cleavage in a biological system such as, for example, the digestive system of an organism or a metabolic system in a living cell.

In some embodiments, biocleavable linking moieties are selected according to their susceptibility to certain enzymes that are likely to be present at the targeted bodily site or at any other bodily site where cleavage is intended, thereby defining the cleavage conditions.

Representative examples of biocleavable moieties include, without limitation, amides, esters, carboxylates, carbamates, phosphates, hydrazides, thiohydrazides, disulfides, epoxides, peroxo and methyleneamines. Such moieties are typically subjected to enzymatic cleavages in a biological system, by enzymes such as, for example, hydrolases, amidases, kinases, peptidases, phospholipases, lipases, proteases, esterases, epoxide hydrolases, nitrilases, glycosidases and the like.

For example, hydrolases (EC number beginning with 3) catalyze hydrolysis of a chemical bond according to the general reaction scheme A−B+H₂O→A−OH+B−H. Ester bonds are cleaved by sub-group of hydrolases known as esterases (EC number beginning with 3.1), which include nucleases, phosphodiesterases, lipases and phosphatases. Hydrolases having an EC number beginning with 3.4 are peptidases, which act on peptide bonds.

Additional information pertaining to enzymes, enzymatic reactions, and enzyme— linking moiety correlations can be found in various publically accessible sources, such as Bairoch A., “The ENZYME database in 2000”, Nucleic Acids Res, 2000, 28, pp. 304-305.

Definitions of specific functional groups, chemical terms, and general terms used throughout the specification are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

### Exemplary Conjugates:

In some embodiments of the present invention, the cyclic peptide may be used to deliver more than one bioactive agent moieties, which may be identical, similar but linked differently (different locations on the peptide or the drug molecule, and/or different linking moieties), or moieties of different bioactive agents.

In some embodiments of the present invention, the linking moiety is part of, or links to a side-chain of one of the amino acid residues in the cyclic peptide. In some embodiments of the present invention, the linking moiety is part of, or links to one of the termini (main-chain) of the cyclic peptide. In some embodiments, the linking moiety is part of, or links as an amide to an amino group of the N-terminus of the peptide. In some embodiments, the linking moiety is part of, or links as an ester or an amide to a carboxy group of the C-terminus of the peptide. In some embodiments, the linking moiety is part of, or links to an amino group of a side-chain of a residue of the peptide. In some embodiments, the linking moiety is part of, or links to a carboxy group of a side-chain of a residue of the peptide. In some embodiments, the linking moiety is part of, or links to a hydroxy group of a side-chain of a residue of the peptide.

Table 2a below presents some exemplary conjugates, according to some embodiments of the present invention, comprising a cyclic peptide moiety, a GABA (y-aminobutyric acid) linking moiety and CPT drug moiety.

TABLE 2a

| Cyclic peptide | PDC Structure |
|---|---|
| P1 (SEQ ID No 1) | 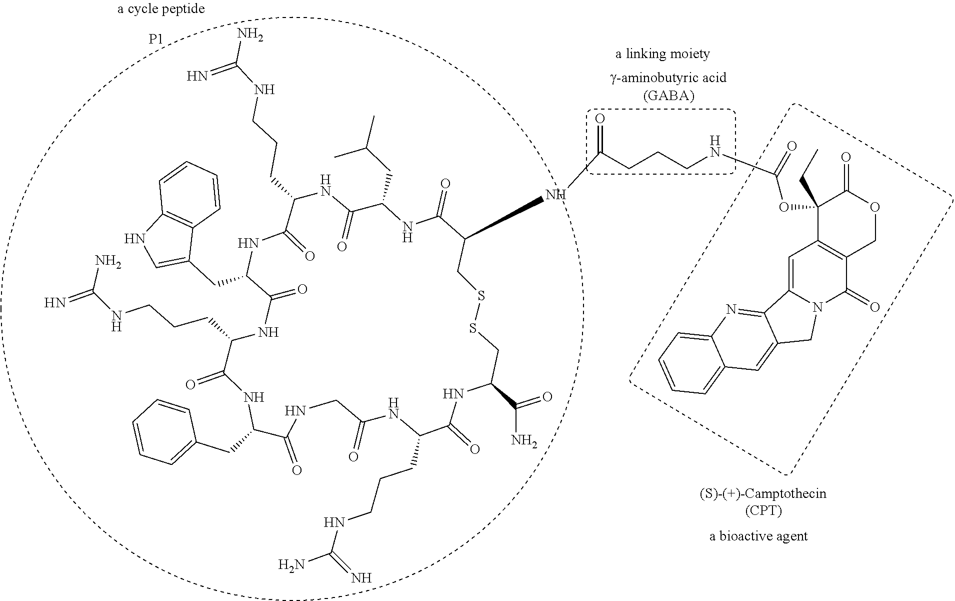 [SMILES: CC[C@@]1(OC(=O)NCCCC(=O)N[C@H]2CSSC[C@@H](C(N)=O)NC(=O)[C@H](CCCNC(=N)N)NC(=O)CNC(=O)[C@H](Cc3ccccc3)NC(=O)[C@H](CCCNC(=N)N)NC(=O)[C@H](Cc3c[nH]c4ccccc34)NC(=O)[C@H](CCCNC(=N)N)NC(=O)[C@H](CC(C)C)NC2=O)C(=O)OCc2c1cc1n(c2=O)Cc2cc3ccccc3nc2-1 ; "P1"] |
| P2 (SEQ ID No 2) | 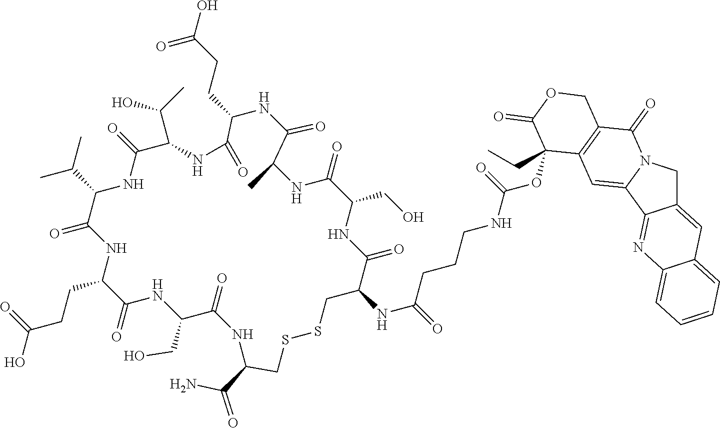 [SMILES: CC[C@@]1(OC(=O)NCCCC(=O)N[C@H]2CSSC[C@@H](C(N)=O)NC(=O)[C@H](CO)NC(=O)[C@H](CCC(=O)O)NC(=O)[C@H](C(C)C)NC(=O)[C@H]([C@@H](C)O)NC(=O)[C@H](CCC(=O)O)NC(=O)[C@H](C)NC(=O)[C@H](CO)NC2=O)C(=O)OCc2c1cc1n(c2=O)Cc2cc3ccccc3nc2-1] |
| P3 (SEQ ID No 3) | 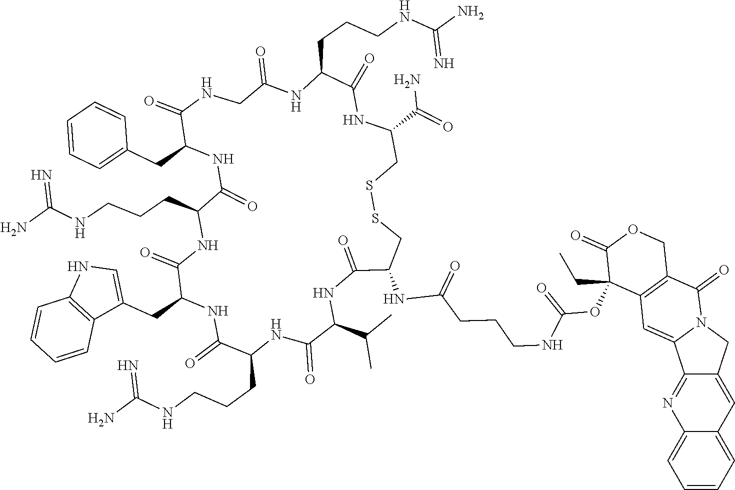 [SMILES: CC[C@@]1(OC(=O)NCCCC(=O)N[C@H]2CSSC[C@@H](C(N)=O)NC(=O)[C@H](CCCNC(=N)N)NC(=O)CNC(=O)[C@H](Cc3ccccc3)NC(=O)[C@H](CCCNC(=N)N)NC(=O)[C@H](Cc3c[nH]c4ccccc34)NC(=O)[C@H](CCCNC(=N)N)NC(=O)[C@H](C(C)C)NC2=O)C(=O)OCc2c1cc1n(c2=O)Cc2cc3ccccc3nc2-1] |
| P4 (SEQ ID No 4) | 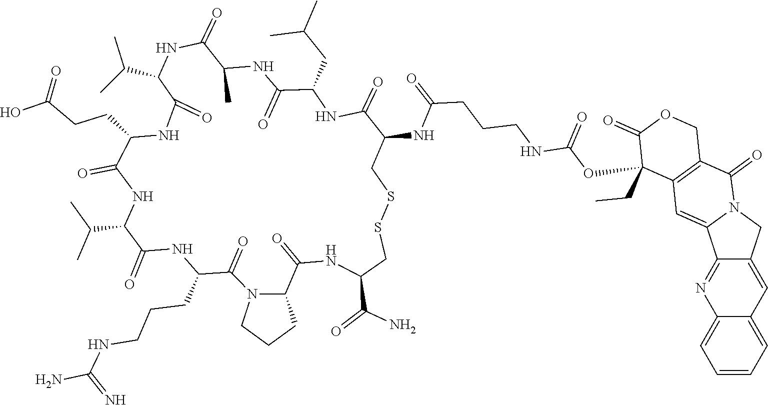 [SMILES: CC[C@@]1(OC(=O)NCCCC(=O)N[C@H]2CSSC[C@@H](C(N)=O)NC(=O)[C@@H]3CCCN3C(=O)[C@H](CCCNC(=N)N)NC(=O)[C@H](C(C)C)NC(=O)[C@H](CCC(=O)O)NC(=O)[C@H](C(C)C)NC(=O)[C@H](C)NC(=O)[C@H](CC(C)C)NC2=O)C(=O)OCc2c1cc1n(c2=O)Cc2cc3ccccc3nc2-1] |
| P5 (SEQ ID No 5) | 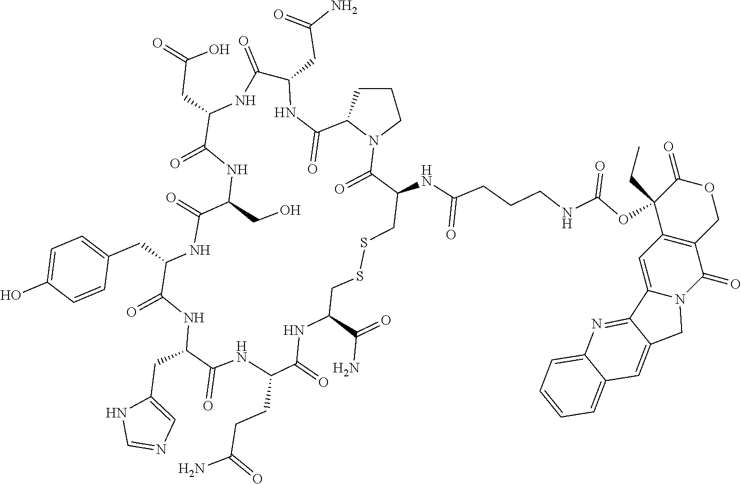 [SMILES: CC[C@@]1(OC(=O)NCCCC(=O)N[C@H]2CSSC[C@@H](C(N)=O)NC(=O)[C@H](CCC(N)=O)NC(=O)[C@H](Cc3cnc[nH]3)NC(=O)[C@H](Cc3ccc(O)cc3)NC(=O)[C@H](CO)NC(=O)[C@H](CC(=O)O)NC(=O)[C@H](CC(N)=O)NC(=O)[C@@H]3CCCN3C2=O)C(=O)OCc2c1cc1n(c2=O)Cc2cc3ccccc3nc2-1] |
| P6 (SEQ ID No 6) | 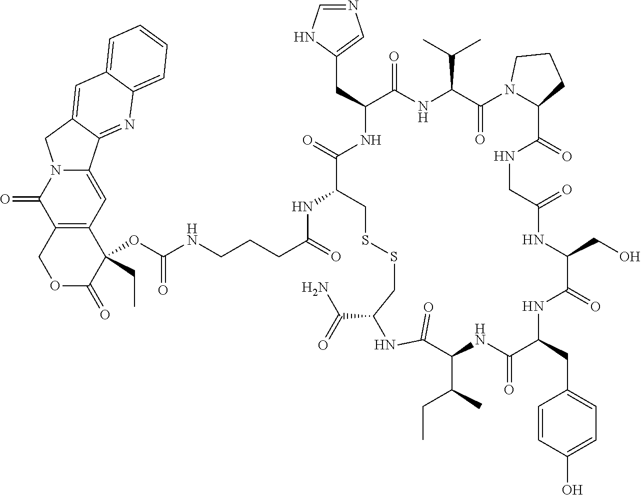 [SMILES: CC[C@H](C)[C@@H]1NC(=O)[C@H](Cc2ccc(O)cc2)NC(=O)[C@H](CO)NC(=O)CNC(=O)[C@@H]2CCCN2C(=O)[C@H](C(C)C)NC(=O)[C@H](Cc2cnc[nH]2)NC(=O)[C@@H](NC(=O)CCCNC(=O)O[C@]2(CC)C(=O)OCc3c2cc2n(c3=O)Cc3cc4ccccc4nc3-2)CSSC[C@@H](C(N)=O)NC1=O] |
| P7 (SEQ ID No 7) | 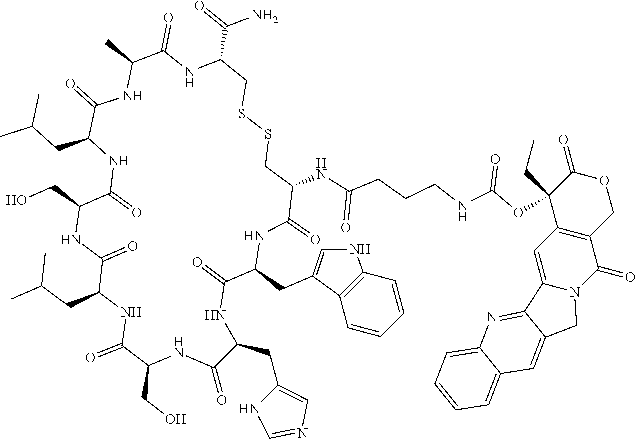 [SMILES: CC[C@@]1(OC(=O)NCCCC(=O)N[C@H]2CSSC[C@@H](C(N)=O)NC(=O)[C@H](C)NC(=O)[C@H](CC(C)C)NC(=O)[C@H](CO)NC(=O)[C@H](CC(C)C)NC(=O)[C@H](CO)NC(=O)[C@H](Cc3cnc[nH]3)NC(=O)[C@H](Cc3c[nH]c4ccccc34)NC2=O)C(=O)OCc2c1cc1n(c2=O)Cc2cc3ccccc3nc2-1] |
| P8 (SEQ ID No 8) | 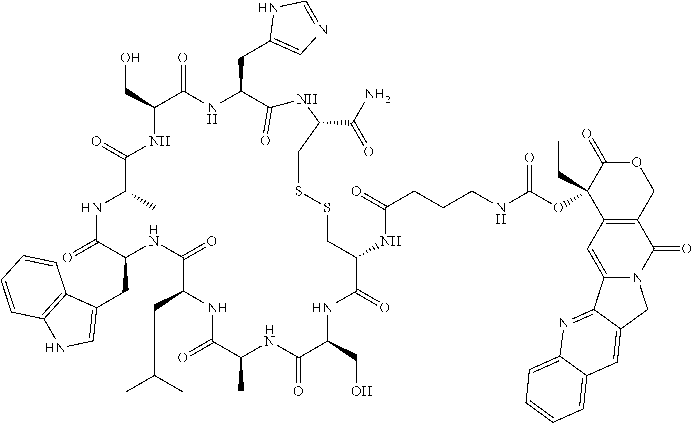 [SMILES: CC[C@@]1(OC(=O)NCCCC(=O)N[C@H]2CSSC[C@@H](C(N)=O)NC(=O)[C@H](Cc3cnc[nH]3)NC(=O)[C@H](CO)NC(=O)[C@H](C)NC(=O)[C@H](Cc3c[nH]c4ccccc34)NC(=O)[C@H](CC(C)C)NC(=O)[C@H](C)NC(=O)[C@H](CO)NC2=O)C(=O)OCc2c1cc1n(c2=O)Cc2cc3ccccc3nc2-1] |
| P9 (SEQ ID No 9) | 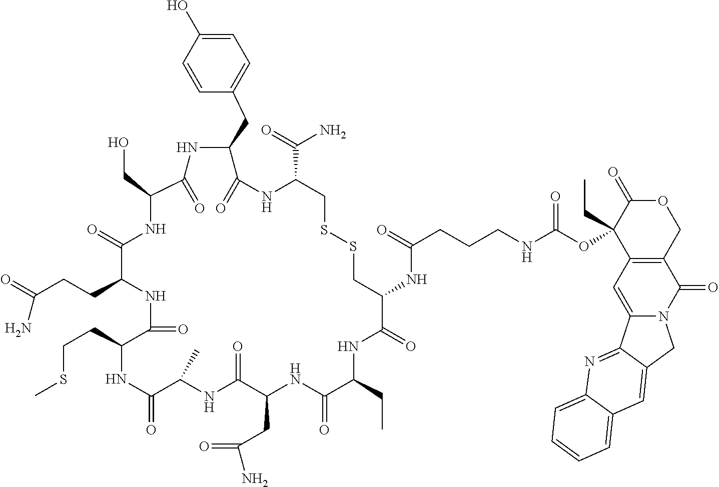 [SMILES: CC[C@@H]1NC(=O)[C@@H](NC(=O)CCCNC(=O)O[C@]2(CC)C(=O)OCc3c2cc2n(c3=O)Cc3cc4ccccc4nc3-2)CSSC[C@@H](C(N)=O)NC(=O)[C@H](Cc2ccc(O)cc2)NC(=O)[C@H](CO)NC(=O)[C@H](CCC(N)=O)NC(=O)[C@H](CCSC)NC(=O)[C@H](C)NC(=O)[C@H](CC(N)=O)NC1=O] |
| P10 (SEQ ID No 10) | 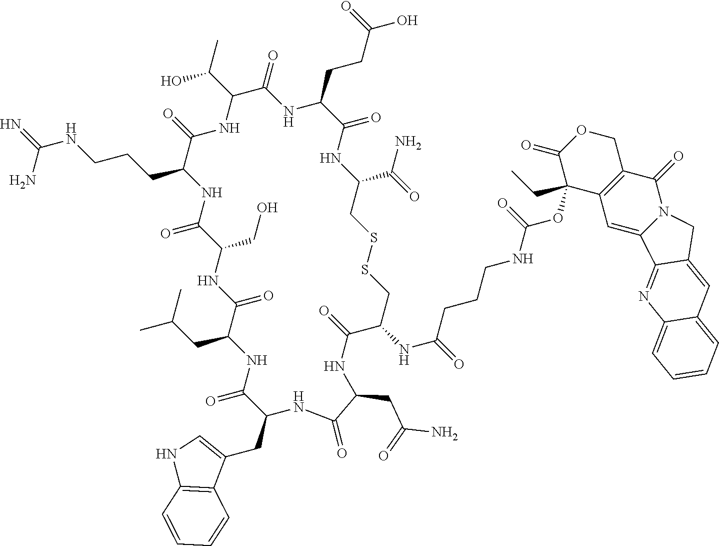 [SMILES: CC[C@@]1(OC(=O)NCCCC(=O)N[C@H]2CSSC[C@@H](C(N)=O)NC(=O)[C@H](CCC(=O)O)NC(=O)C([C@@H](C)O)NC(=O)[C@H](CCCNC(=N)N)NC(=O)[C@H](CO)NC(=O)[C@H](CC(C)C)NC(=O)[C@H](Cc3c[nH]c4ccccc34)NC(=O)[C@H](CC(N)=O)NC2=O)C(=O)OCc2c1cc1n(c2=O)Cc2cc3ccccc3nc2-1] |
| P11 (SEQ ID No 11) | 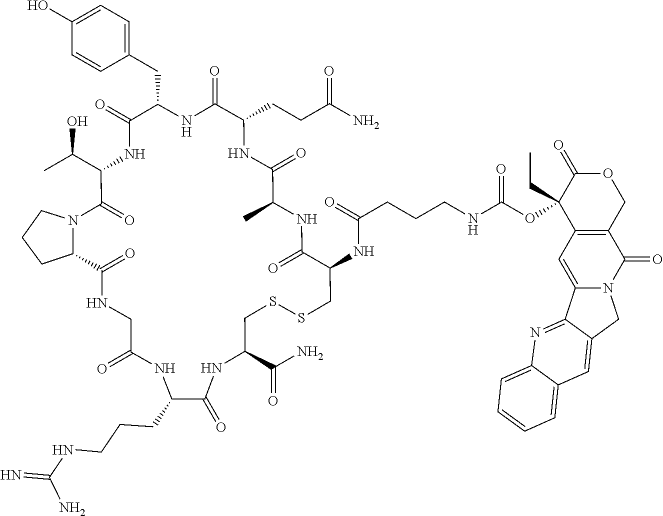 [SMILES: CC[C@@]1(OC(=O)NCCCC(=O)N[C@H]2CSSC[C@@H](C(N)=O)NC(=O)[C@H](CCCNC(=N)N)NC(=O)CNC(=O)[C@@H]3CCCN3C(=O)[C@H]([C@@H](C)O)NC(=O)[C@H](Cc3ccc(O)cc3)NC(=O)[C@H](CCC(N)=O)NC(=O)[C@H](C)NC2=O)C(=O)OCc2c1cc1n(c2=O)Cc2cc3ccccc3nc2-1] |

Table 2b below presents some exemplary conjugates, according to some embodiments of the present invention, comprising a cyclic peptide moiety, a GABA (γ-aminobutyric acid) linking moiety and the labeling moiety derived from fluorescein 5-isothiocyanate (FITC).

TABLE 2b

| Cyclic peptide | PDC Structure |
|---|---|
| P1 (SEQ ID No 1) | 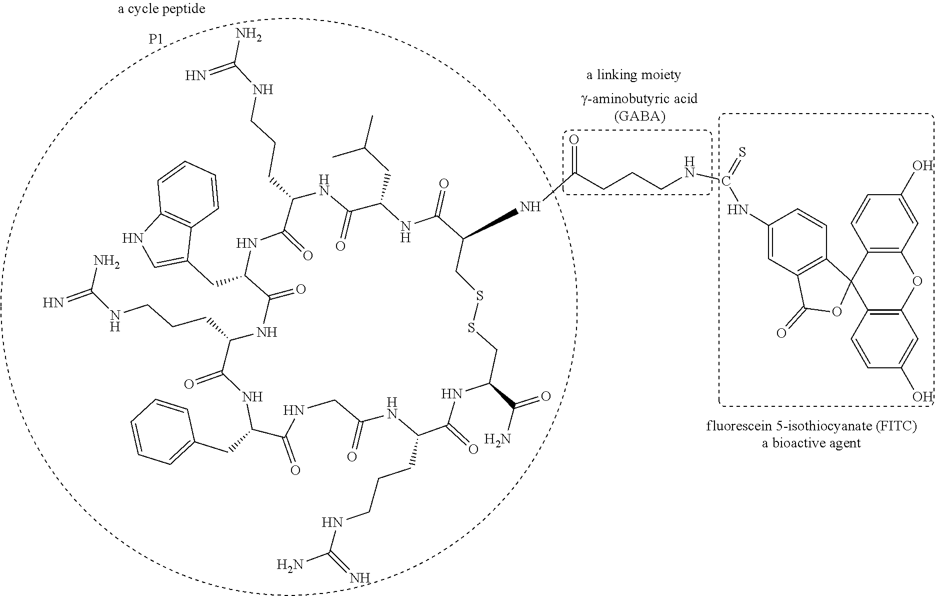 [SMILES: CC(C)C[C@@H]1NC(=O)[C@@H](NC(=O)CCCNC(=S)Nc2ccc3c(c2)C(=O)OC32c3ccc(O)cc3Oc3cc(O)ccc32)CSSC[C@@H](C(N)=O)NC(=O)[C@H](CCCNC(=N)N)NC(=O)CNC(=O)[C@H](Cc2ccccc2)NC(=O)[C@H](CCCNC(=N)N)NC(=O)[C@H](Cc2c[nH]c3ccccc23)NC(=O)[C@H](CCCNC(=N)N)NC1=O ; "P1"] |
| P2 (SEQ ID No 2) | 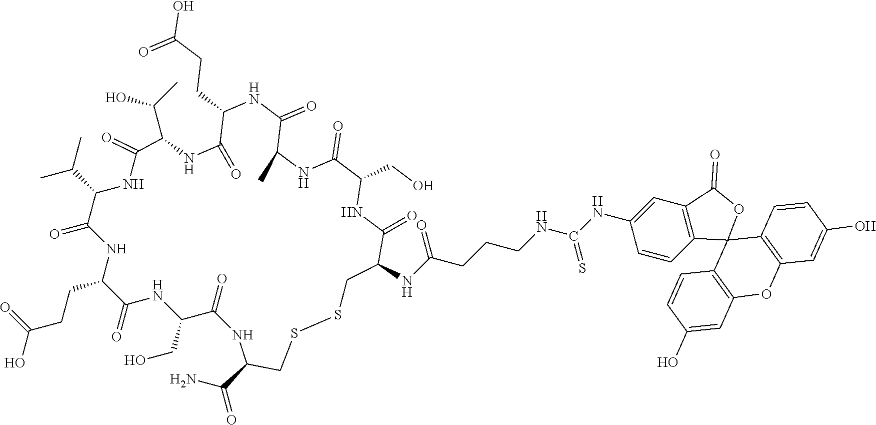 [SMILES: CC(C)[C@@H]1NC(=O)[C@H]([C@@H](C)O)NC(=O)[C@H](CCC(=O)O)NC(=O)[C@H](C)NC(=O)[C@H](CO)NC(=O)[C@@H](NC(=O)CCCNC(=S)Nc2ccc3c(c2)C(=O)OC32c3ccc(O)cc3Oc3cc(O)ccc32)CSSC[C@@H](C(N)=O)NC(=O)[C@H](CO)NC(=O)[C@H](CCC(=O)O)NC1=O] |
| P3 (SEQ ID No 3) | 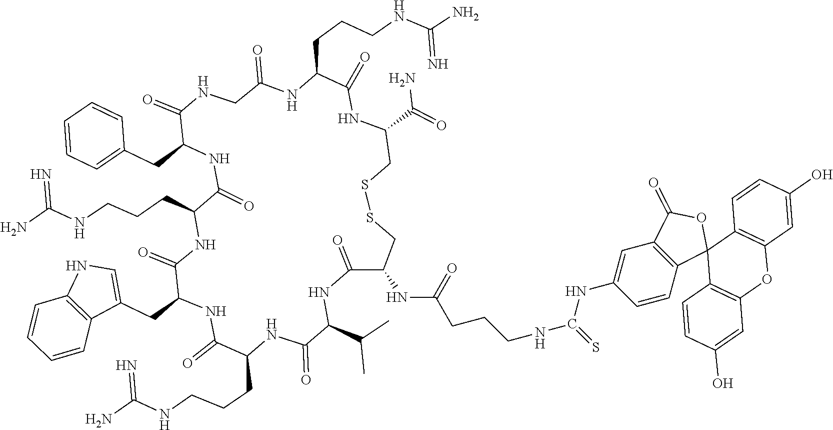 [SMILES: CC(C)[C@@H]1NC(=O)[C@@H](NC(=O)CCCNC(=S)Nc2ccc3c(c2)C(=O)OC32c3ccc(O)cc3Oc3cc(O)ccc32)CSSC[C@@H](C(N)=O)NC(=O)[C@H](CCCNC(=N)N)NC(=O)CNC(=O)[C@H](Cc2ccccc2)NC(=O)[C@H](CCCNC(=N)N)NC(=O)[C@H](Cc2c[nH]c3ccccc23)NC(=O)[C@H](CCCNC(=N)N)NC1=O] |
| P4 (SEQ ID No 4) | 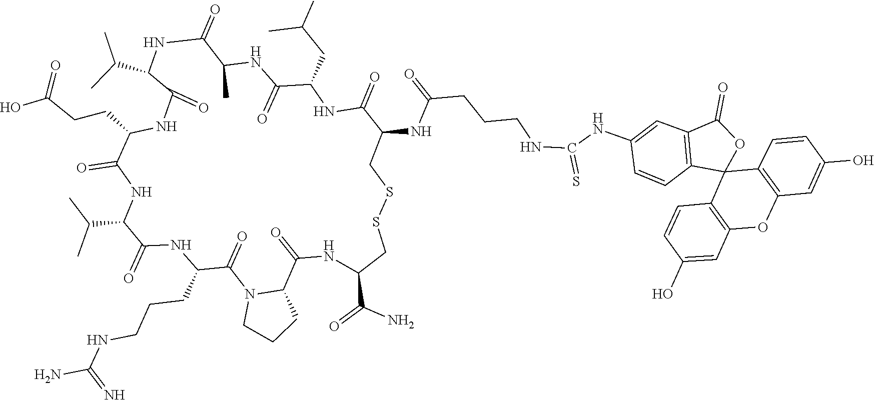 [SMILES: CC(C)C[C@@H]1NC(=O)[C@@H](NC(=O)CCCNC(=S)Nc2ccc3c(c2)C(=O)OC32c3ccc(O)cc3Oc3cc(O)ccc32)CSSC[C@@H](C(N)=O)NC(=O)[C@@H]2CCCN2C(=O)[C@H](CCCNC(=N)N)NC(=O)[C@H](C(C)C)NC(=O)[C@H](CCC(=O)O)NC(=O)[C@H](C(C)C)NC(=O)[C@H](C)NC1=O] |
| P5 (SEQ ID No 5) | 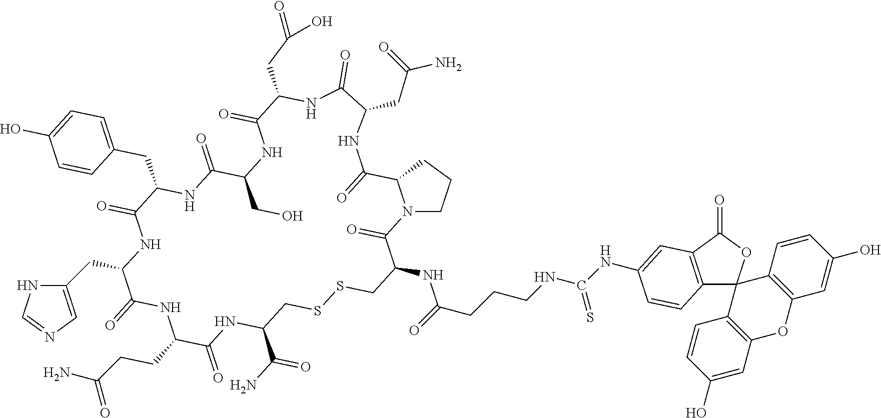 [SMILES: NC(=O)CC[C@@H]1NC(=O)[C@H](Cc2cnc[nH]2)NC(=O)[C@H](Cc2ccc(O)cc2)NC(=O)[C@H](CO)NC(=O)[C@H](CC(=O)O)NC(=O)[C@H](CC(N)=O)NC(=O)[C@@H]2CCCN2C(=O)[C@@H](NC(=O)CCCNC(=S)Nc2ccc3c(c2)C(=O)OC32c3ccc(O)cc3Oc3cc(O)ccc32)CSSC[C@@H](C(N)=O)NC1=O] |
| P6 (SEQ ID No 6) | 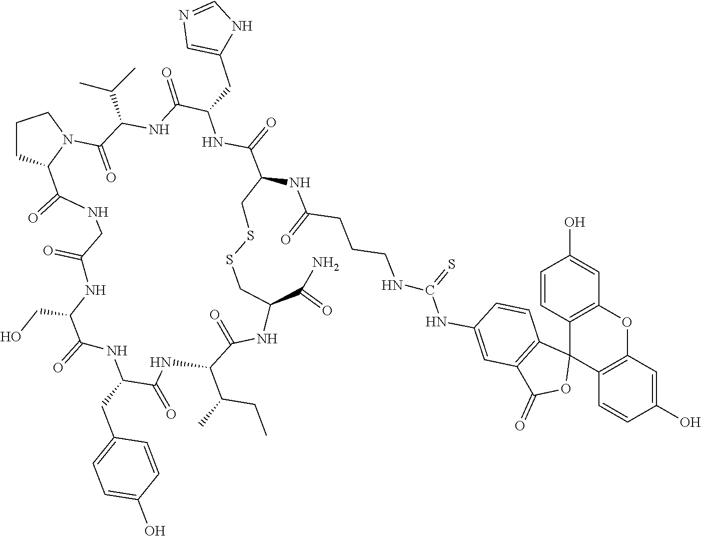 [SMILES: CC[C@H](C)[C@@H]1NC(=O)[C@H](Cc2ccc(O)cc2)NC(=O)[C@H](CO)NC(=O)CNC(=O)[C@@H]2CCCN2C(=O)[C@H](C(C)C)NC(=O)[C@H](Cc2cnc[nH]2)NC(=O)[C@@H](NC(=O)CCCNC(=S)Nc2ccc3c(c2)C(=O)OC32c3ccc(O)cc3Oc3cc(O)ccc32)CSSC[C@@H](C(N)=O)NC1=O] |
| P7 (SEQ ID No 7) | 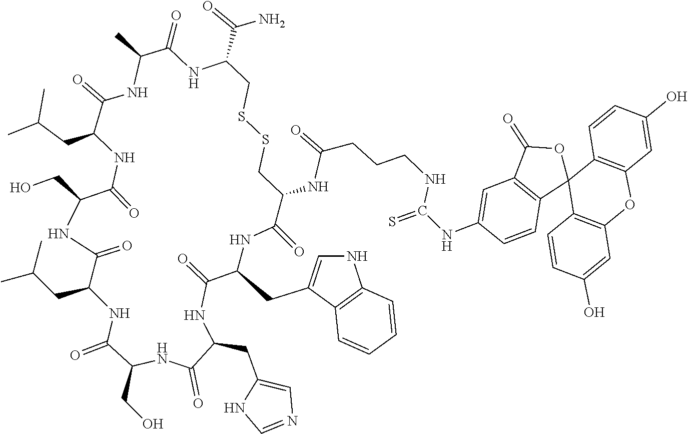 [SMILES: CC(C)C[C@@H]1NC(=O)[C@H](CO)NC(=O)[C@H](CC(C)C)NC(=O)[C@H](CO)NC(=O)[C@H](Cc2cnc[nH]2)NC(=O)[C@H](Cc2c[nH]c3ccccc23)NC(=O)[C@@H](NC(=O)CCCNC(=S)Nc2ccc3c(c2)C(=O)OC32c3ccc(O)cc3Oc3cc(O)ccc32)CSSC[C@@H](C(N)=O)NC(=O)[C@H](C)NC1=O] |
| P8 (SEQ ID No 8) | 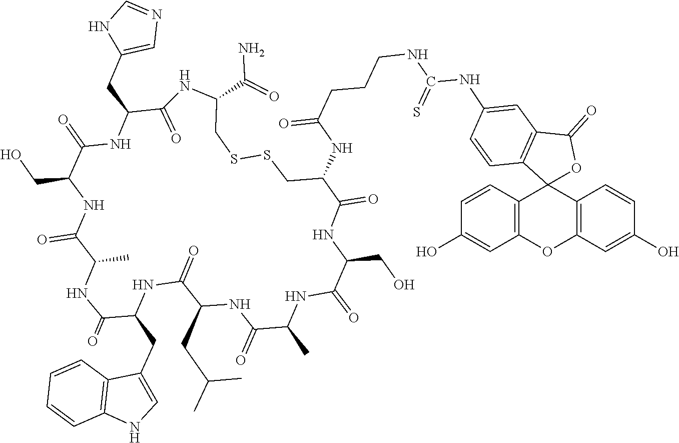 [SMILES: CC(C)C[C@@H]1NC(=O)[C@H](C)NC(=O)[C@H](CO)NC(=O)[C@@H](NC(=O)CCCNC(=S)Nc2ccc3c(c2)C(=O)OC32c3ccc(O)cc3Oc3cc(O)ccc32)CSSC[C@@H](C(N)=O)NC(=O)[C@H](Cc2cnc[nH]2)NC(=O)[C@H](CO)NC(=O)[C@H](C)NC(=O)[C@H](Cc2c[nH]c3ccccc23)NC1=O] |
| P9 (SEQ ID No 9) | 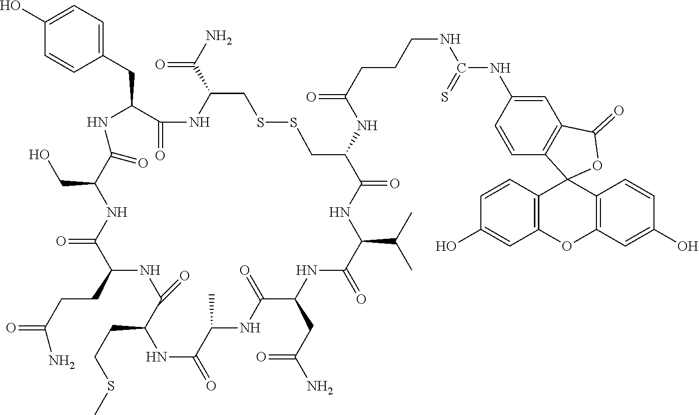 [SMILES: CSCC[C@@H]1NC(=O)[C@H](C)NC(=O)[C@H](CC(N)=O)NC(=O)[C@H](C(C)C)NC(=O)[C@@H](NC(=O)CCCNC(=S)Nc2ccc3c(c2)C(=O)OC32c3ccc(O)cc3Oc3cc(O)ccc32)CSSC[C@@H](C(N)=O)NC(=O)[C@H](Cc2ccc(O)cc2)NC(=O)[C@H](CO)NC(=O)[C@H](CCC(N)=O)NC1=O] |
| P10 (SEQ ID No 10) | 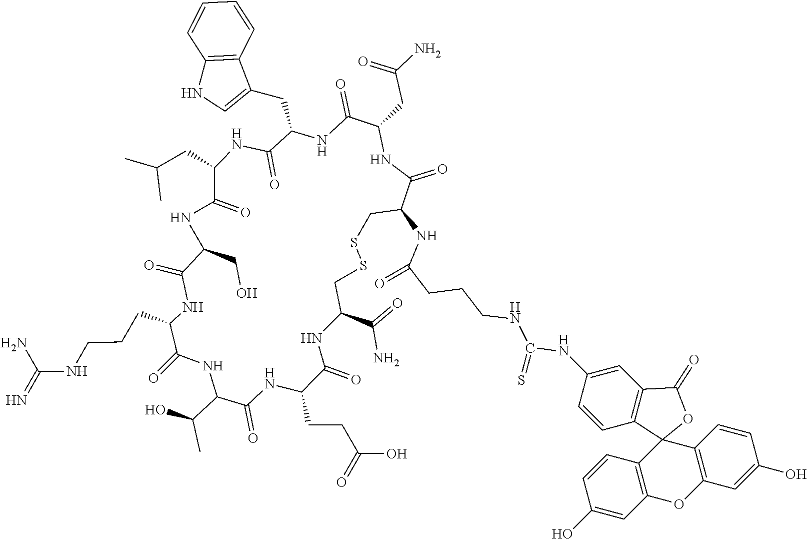 [SMILES: CC(C)C[C@@H]1NC(=O)[C@H](Cc2c[nH]c3ccccc23)NC(=O)[C@H](CC(N)=O)NC(=O)[C@@H](NC(=O)CCCNC(=S)Nc2ccc3c(c2)C(=O)OC32c3ccc(O)cc3Oc3cc(O)ccc32)CSSC[C@@H](C(N)=O)NC(=O)[C@H](CCC(=O)O)NC(=O)C([C@@H](C)O)NC(=O)[C@H](CCCNC(=N)N)NC(=O)[C@H](CO)NC1=O] |
| P11 (SEQ ID No 11) | 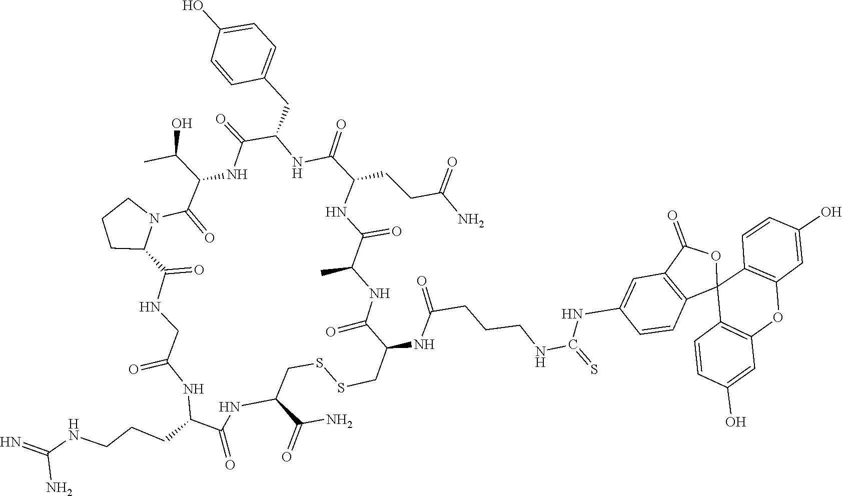 [SMILES: C[C@@H]1NC(=O)[C@@H](NC(=O)CCCNC(=S)Nc2ccc3c(c2)C(=O)OC32c3ccc(O)cc3Oc3cc(O)ccc32)CSSC[C@@H](C(N)=O)NC(=O)[C@H](CCCNC(=N)N)NC(=O)CNC(=O)[C@@H]2CCCN2C(=O)[C@H]([C@@H](C)O)NC(=O)[C@H](Cc2ccc(O)cc2)NC(=O)[C@H](CCC(N)=O)NC1=O] |

Additional examples of PDCs, according to embodiments of the present invention, include, without limitation, a cyclic peptide conjugated via its N-terminal amine and one or more linking moieties, such as GABA, glutathione, lysine, succinic acid, 2-amino-5-(carbamoylamino) pentanoic acid (para-aminobenzoic acid; PABA), valine-citrulline-PABA, citrulline, and/or 3-((2-aminoethyl)disulfanyl) propanoic acid, to a drug moiety, such as doxorubicin (DOX), monomethyl auristatin F (MMFA), 7-ethyl-10-hydroxycamptothecin (SN38; an analogue of CPT), and/or a labeling moieties, such as cyanine (Cy5), sulfo-cyanine5 or iodinated xanthene-cyanine NIR dyes, as illustrated in the schemes bellow.

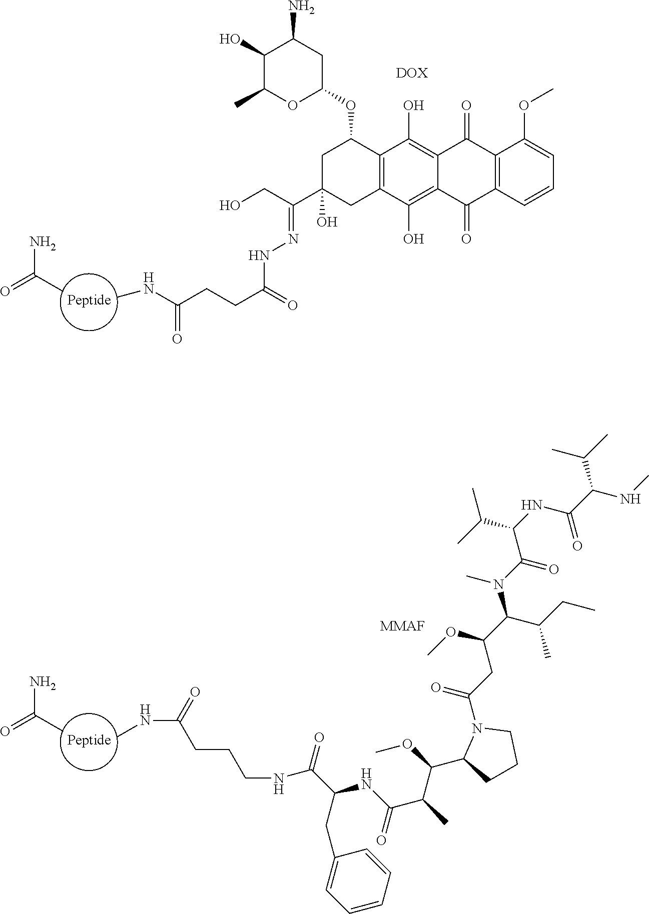

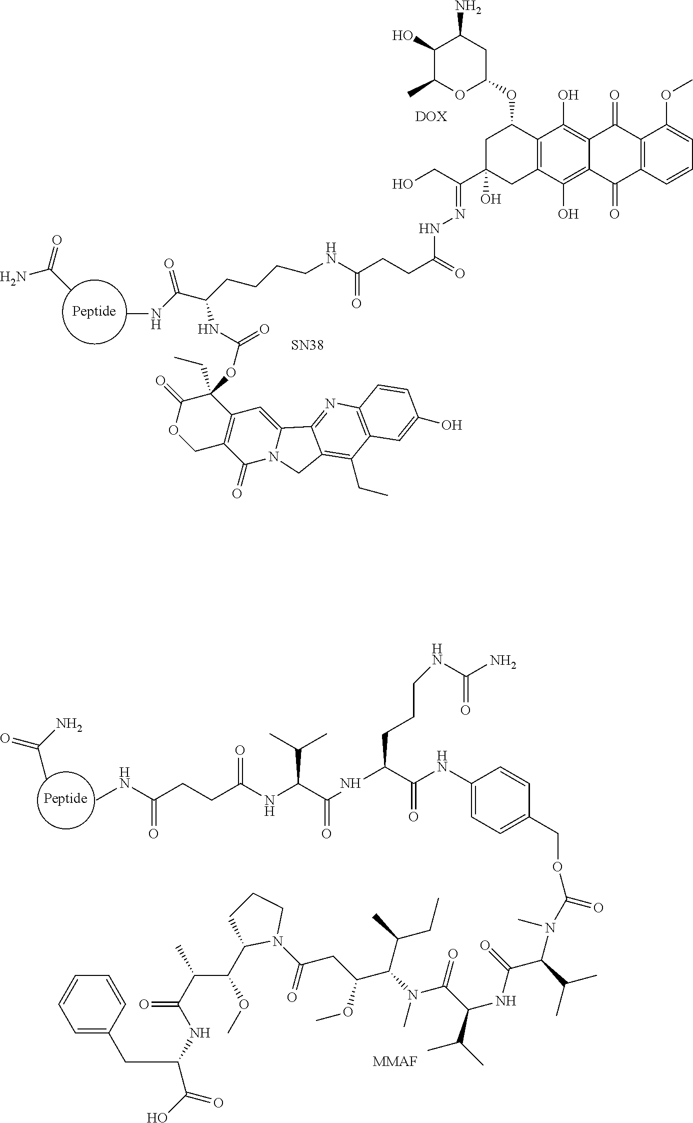

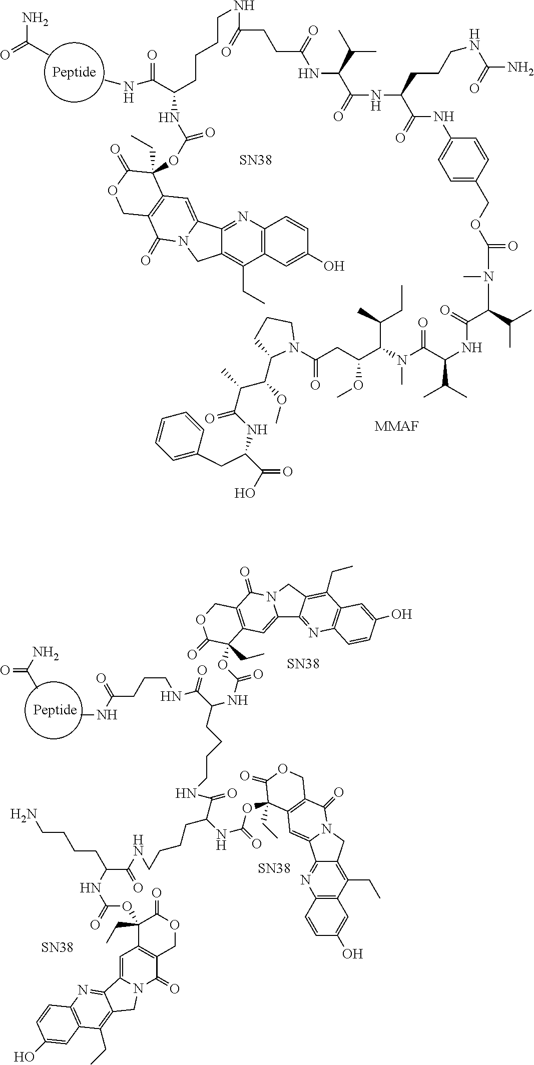

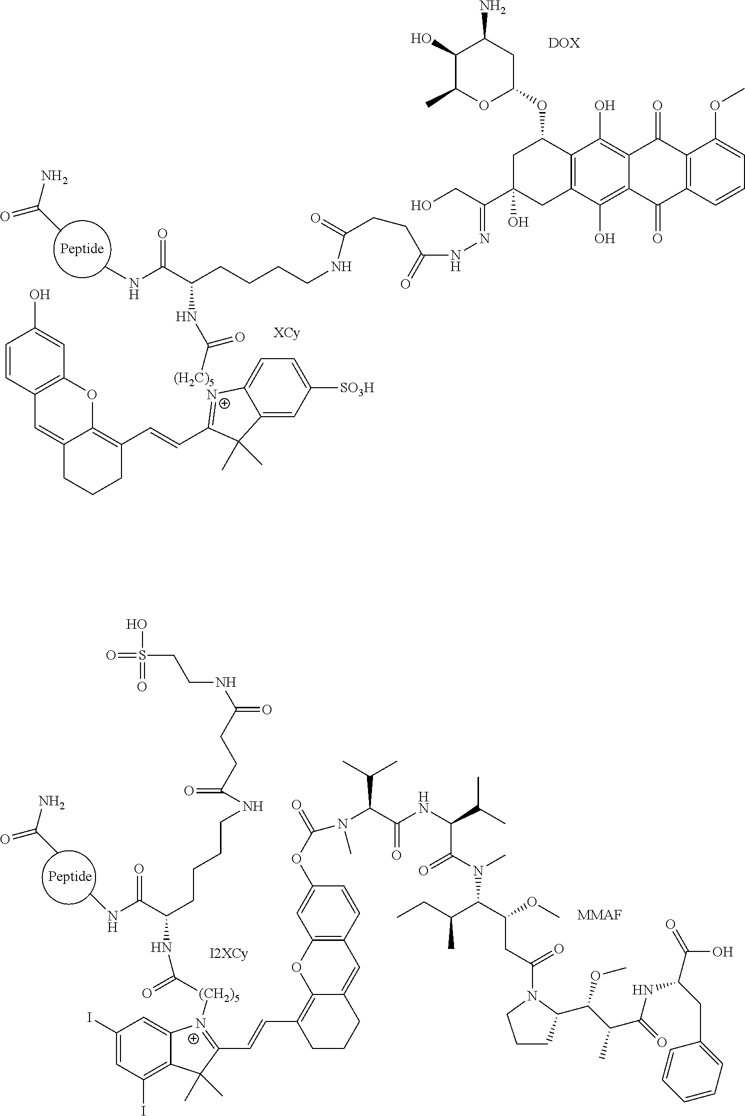

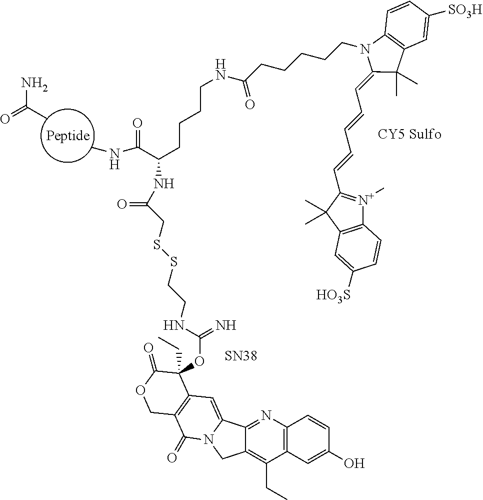

### Practical Uses and Applications

The family of cyclic 7-mer plus two terminal cysteine peptides (total 9-mer cyclic peptides), that are specific to EGFR-overexpressed H1299 (also known as NCI-H1299 or CRL-5803) and its mutation EGRFvIII-expressed DKMG cell lines, and which have been discovered using combined phage display cell-based positive biopanning selection method followed by NGS sequencing of the collected DNA phages, have been shown to be active in vivo, as presented in the Examples section that follows.

At least two exemplary candidates of these peptide family, P6 and P9, have been tested for therapeutic properties that are critical for a peptide-drug conjugate, and exhibited an effective binding and internalization to EGFR and EGFRvIII cell lines as confirmed using FACS and fluorescent microscopy. Moreover, P6 and P9 conjugated to CPT enabled nuclear accumulation of cytotoxic CPT. CPT intracellular accumulation results in DNA damage and induction of cell death. Hence, coupling of CPT to the cyclic peptides provided herein did not interfere with CPT cytotoxic effects, while contributing a specific targeting tool to EGFR and EGFRvIII overexpressed cell lines, namely cancer cells.

Docking simulations reviled that the peptides interact with the EGFR receptor in vicinity to the EGF binding site showing substantial overlap with EGF. Taking all together, it is shown that the presently provided cyclic peptides are excellent carriers for drug delivery to EGFR overexpressed cancers and its EGFRvIII mutation.

Furthermore, since the cyclic peptides provided herein have been shown to be minimally cytotoxic and even non-cytotoxic as well as to efficiently bind to EGFR, one or more of the cyclic peptides provided herein, as well as salts or esters thereof, alone or in combination of one or more additional cyclic peptides, is useful for use as an active ingredient in a medicament, or as a medicament for the treatment of cancer.

It is shown that the cyclic peptides provided herein can deliver a diagnostic and/or therapeutic payload to EGFR-positive cells, such as cancer cells.

### A Pharmaceutical Composition:

Thus, according to an aspect of some embodiments of the present invention, there is provided a pharmaceutical composition, which includes as an active ingredient, the peptide-drug conjugate as provided and demonstrated herein. Similarly, there is provided a use of the conjugates, according to embodiments of the present invention, in the preparation of a medicament. According to some embodiments of the present invention, the pharmaceutical composition or medicament, are used to treat a medical condition treatable by at least one of the drugs linked and controllably releasable from the peptide-drug conjugate.

In some of any of the respective embodiments of the present invention, the pharmaceutical composition or medicament is packaged in a packaging material and identified in print, in or on the packaging material, for use in the treatment of a medical condition or a symptom associated with EGFR-positive cells, and/or for use in the treatment of a medical condition treatable by the drug(s) linked and controllably releasable from the peptide-drug conjugate, and/or for use in treating cancer. The conjugate, according to some embodiments of the present invention, may be incorporated into any suitable pharmaceutically acceptable carrier prior to use.

The conjugate may be administered by any conventional approach known and/or used in the art. In any of the uses described herein, the PDC provided herein can be administered as a part of a pharmaceutical composition, which further comprises a pharmaceutical acceptable carrier, as detailed hereinbelow. The carrier is selected suitable to the selected route of administration.

The PDCs presented herein can be administered via any administration route, including, but not limited to, orally, by inhalation, or parenterally, for example, by intravenous drip or intraperitoneal, subcutaneous, intramuscular or intravenous injection, or topically (including ophthalmically, vaginally, rectally, intranasally).

The formulations for medical use of the conjugate according to the present embodiments, typically include such agents in association with a pharmaceutically acceptable carrier, and optionally other therapeutic ingredient(s). The carrier(s) should be “acceptable” in the sense of being compatible with the other ingredients of the formulations and not deleterious to the recipient thereof. Pharmaceutically acceptable carriers, in this regard, are intended to include any and all solvents, dispersion media, coatings, antibacterial and antifungal agents, isotonic and absorption delaying agents, and the like, compatible with pharmaceutical administration. The use of such media and agents for pharmaceutically active substances is known in the art. Except insofar as any conventional media or agent is incompatible with the active ligand, use thereof in the compositions is contemplated. Supplementary active agents, identified or designed according to the invention and/or known in the art, also can be incorporated into the compositions. The formulations may conveniently be presented in dosage unit form and may be prepared by any of the methods well known in the art of pharmacology/microbiology. In general, some formulations are prepared by bringing the active ligand into association with a liquid carrier or a finely divided solid carrier or both, and then, if necessary, shaping the product into the desired formulation.

Pharmaceutical compositions according to some embodiments of the present invention are formulated to be compatible with its intended route of administration. Solutions or suspensions used for the herein intended application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Pharmaceutical compositions suitable for injectable use, according to some embodiments of the present invention, include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS).

Other than the conjugate and a pharmaceutically acceptable carrier, the pharmaceutical composition may also include other agents that have an effect on nerve cells, such as agents producing peripheral axonopathies.

### Method of Treatment:

Also provided herein is a method of treating a medical condition in a subject in need thereof, which includes administering to the subject a therapeutically effective amount of the cyclic peptide-drug conjugate provided herein, according to embodiments of the present invention.

As used herein, the phrase “therapeutically effective amount” describes an amount of an active agent or a cyclic peptide-drug conjugate being administered, which will relieve to some extent one or more of the symptoms of the medical condition being treated. In the context of the present embodiments, the phrase “therapeutically effective amount” describes an amount of a cyclic peptide-drug conjugate being administered and/or re-administered, which will relieve to some extent one or more of the symptoms of the condition being treated by being at a level that is harmful to the target cell(s) or microorganism(s), and cause a disruption to the life-cycle of the target cell(s) or microorganism(s).

In the context of embodiments of the present invention, the therapeutically effective amount may refer to the cyclic peptide-drug conjugate as a whole or to the amount of one or more bioactive agent releasably attached thereto. The efficacy of any bioactive agent, including the cyclic peptide-drug conjugates presented herein, can be determined by several methodologies known in the art.

According to another aspect of embodiments of the present invention, any one of the cyclic peptide-drug conjugates described herein is identified for use in treating a subject diagnosed with a medical condition treatable by at least one of the drugs linked and controllably releasable from the peptide-drug conjugate.

According to another aspect of embodiments of the present invention, there is provided a use of any of the peptide-drug conjugates described herein as a medicament or as an ingredient in a medicament. In some embodiments, the medicament is for treating a subject diagnosed with a medical condition treatable by at least one of the drugs linked and controllably releasable from the peptide-drug conjugate.

### Anticancer Treatment:

To date, chemotherapy remains the most common and most frequently used in cancer treatment, alone or in combination with other therapies. Currently available anticancer chemotherapies act by affecting specific molecular targets in proliferating cancer cells, leading to inhibition of essential intracellular processes such as DNA transcription, synthesis and replication.

Unfortunately, anticancer drugs are highly toxic, as they are designed to kill mammalian cells, and are therefore harmful also to normal proliferating cells resulting in debilitating and even lethal side effects. Some of these adverse effects are gastrointestinal toxicity, nausea, vomiting, and diarrhea when the epithelial lining of the intestine is affected. Other side effects include alopecia, when the hair follicles are attacked, bone marrow suppression and neutropenia due to toxicity of hematopoietic precursors. Therefore, the effectiveness of currently used anticancer drugs is dose-limited due to their toxicity to normal rapidly growing cells. The peptide-drug conjugate presented herein can be used to treat any medical condition that is treatable by peptide-drug conjugate, by administration of a therapeutically effective amount of the peptide-drug conjugate to a subject in need thereof. The peptide-drug conjugate, according to some embodiments of the present invention, can also be used to prepare a pharmaceutical composition comprising the same and a pharmaceutically acceptable carrier and other optional ingredients. Thus, the peptide-drug conjugate provided herein can be used as an active ingredient in a method of treating any medical condition that is treatable by peptide-drug conjugate, by administering a therapeutically effective amount thereof to the subject in need thereof.

The peptide-drug conjugate presented herein can be used to treat any medical condition that is treatable by administration of a bioactive agent (drug) in the context of a peptide-drug conjugate or the analog thereof, according to some embodiments of the present invention. According to some embodiments of the present invention, it is advantageous to use the peptide-drug conjugate to treat medical conditions, which are treatable by administration of a combination of drugs. In some embodiments, the medical condition includes cancer, and more specifically, cancer of the type that is characterized by EGFR-positive cells. In the context of the present invention, EGFR-positive cells are cells that have high levels of expression of the epidermal growth factor receptor (EGFR) or mutants thereof, which is a transmembrane receptor protein that is activated by the binding of epidermal growth factor (EGF) or other related ligands. These receptors are involved in cell signaling pathways that control cell proliferation, differentiation, and survival. Overexpression or activation of EGFR or mutations thereof has been associated with the development and progression of various types of cancer, and drugs that target EGFR are used in cancer therapy. EGFR-positive cells are often studied in cancer research to better understand the role of EGFR in tumor development and to develop new drugs that can specifically target these cells.

The peptide-drug conjugates provided herein, according to some embodiments of the present invention, allow controlled and cell-specific anticancer activity. In some embodiments of the present invention, the medical condition is associated with malignant cells and tumors, collectively referred to herein as cancer.

The use of a conjugate according to embodiments of the present invention, can optimize the balance between the desired anticancer activity of certain anticancer drugs and their adverse side effects, by quantitative determination of the actual amount of drug released in the targeted cells.

In some embodiments, the functional moiety of the peptide-drug conjugates presented herein, is the cyclic peptide moiety that is responsible for the higher concentration of the peptide-drug conjugate at the targeted bodily site compared to non-targeted bodily sites, thereby reducing the adverse side effects associated with the toxicity of the anti-cancer drugs attached thereto. In addition, the linking moieties conjugating the anticancer drug to the cyclic peptide are selected such that they cleave in conditions that are present at the targeted site more so than in non-targeted sites, thereby releasing the payload of drugs at the targeted site at a higher rate compared to non-targeted sites.

In the context of some embodiments of the present invention, the term “cancer” refers, but not limited to acute lymphoblastic, acute lymphoblastic leukemia, acute lymphocytic leukemia, acute myelogenous leukemia, acute myeloid leukemia, adrenocortical carcinoma, AIDS-related lymphoma, anal cancer, appendix cancer, basal-cell carcinoma, bladder cancer, brain cancer, brainstem glioma, breast cancer, bronchial adenomas/carcinoids, Burkitt's lymphoma, carcinoid tumor, cerebellar or cerebral astrocytoma, cervical cancer, cholangiocarcinoma, chondrosarcoma, chronic lymphocytic or chronic lymphocytic leukemia, chronic myelogenous or chronic myeloid leukemia, chronic myeloproliferative disorders, colon cancer, cutaneous T-cell lymphoma, desmoplastic small round cell tumor, endometrial uterine cancer, ependymoma, esophageal cancer, Ewing's sarcoma, extracranial germ cell tumor, extragonadal germ cell tumor, extrahepatic bile duct cancer, gallbladder cancer, gastric (stomach) cancer, gastrointestinal carcinoid tumor, gastrointestinal stromal tumor (GIST), gestational trophoblastic tumor, glioma of the brain stem, hairy cell leukemia, head and neck cancer, heart cancer, hepatocellular (liver) cancer, Hodgkin lymphoma, hypopharyngeal cancer, hypothalamic and visual pathway glioma, intraocular melanoma, Islet cell carcinoma, Kaposi sarcoma, laryngeal cancer, leukaemia, lip and oral cavity cancer, liposarcoma, lymphoma, male breast cancer, malignant mesothelioma, medulloblastoma, melanoma, Merkel cell skin carcinoma, mesothelioma, metastatic squamous neck cancer, mouth cancer, multiple endocrine neoplasia syndrome, multiple myeloma, multiple myeloma/plasma cell neoplasm, mycosis fungoides, myelodysplastic/myeloproliferative diseases, nasal cavity and paranasal sinus cancer, nasopharyngeal carcinoma, neuroblastoma, non-Hodgkin lymphoma, non-melanoma skin cancer, non-small cell lung cancer, oligodendroglioma, oral cancer, oropharyngeal cancer, osteosarcoma and malignant fibrous histiocytoma, ovarian cancer, ovarian germ cell tumor, ovarian epithelial cancer (surface epithelial-stromal tumor), ovarian low malignant potential tumor, pancreatic cancer, paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, pituitary adenoma, plasma cell neoplasia, pleuropulmonary blastoma, primary carcinoma, primary central nervous system lymphoma, primary liver cancer, prostate cancer, rectal cancer, renal cell carcinoma, renal pelvis and ureter carcinoma, retinoblastoma, rhabdomyosarcoma, salivary gland cancer, Sézary syndrome, small cell lung cancer, small intestine cancer, soft tissue sarcoma, squamous cell carcinoma, stomach cancer, supratentorial primitive neuroectodermal tumor, testicular cancer, throat cancer, thymoma and thymic carcinoma, thyroid cancer, transitional cell cancer of the renal pelvis and ureter, urethral cancer, uterine sarcoma, vaginal cancer, visual pathway and hypothalamic glioma, vulvar cancer, Waldenström macroglobulinemia and Wilms tumor.

The peptide-drug conjugates provided herein, according to some embodiments of the present invention, can be used in basic research where specific cancer cell targeting is required.

### Chemical Definitions

Definitions of specific functional groups, chemical terms, and general terms used throughout the specification are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

As used herein, the terms “amine” or “amino”, describe both a —NR′R″ end group and a —NR′— linking moiety, wherein R′ and R″ are each independently hydrogen, alkyl, cycloalkyl, aryl, as these terms are defined hereinbelow.

The amine group can therefore be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

Alternatively, R′ and R″ can each independently be hydrogen, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine, as these terms are defined herein.

The term “alkyl” describes a saturated aliphatic hydrocarbon including straight chain (unbranched) and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or unsubstituted. Substituted alkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

The alkyl group can be an end group, as this phrase is defined hereinabove, wherein it is attached to a single adjacent atom, or a linking moiety, as this phrase is defined hereinabove, which connects two or more moieties via at least two carbons in its chain. When an alkyl is a linking moiety, it is also referred to herein as “alkylene”, e.g., methylene, ethylene, propylene, etc.

The term “alkenyl” describes an unsaturated alkyl, as defined herein, having at least two carbon atoms and at least one carbon-carbon double bond. The alkenyl may be substituted or unsubstituted by one or more substituents, as described for alkyl hereinabove.

The terms “alkynyl” or “alkyne”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e., rings that share an adjacent pair of carbon atoms) group where one or more of the rings does not have a completely conjugated pi-electron system. The cycloalkyl group may be substituted or unsubstituted. Substituted cycloalkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The cycloalkyl group can be an end group, as this phrase is defined hereinabove, wherein it is attached to a single adjacent atom, or a linking moiety, as this phrase is defined hereinabove, connecting two or more moieties at two or more positions thereof.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. Substituted heteroalicyclic may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroalicyclic group can be an end group, as this phrase is defined hereinabove, where it is attached to a single adjacent atom, or a linking moiety, as this phrase is defined hereinabove, connecting two or more moieties at two or more positions thereof. Representative examples are piperidine, piperazine, tetrahydrofurane, tetrahydropyrane, morpholino and the like.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted. Substituted aryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The aryl group can be an end group, as this term is defined hereinabove, wherein it is attached to a single adjacent atom, or a linking moiety, as this term is defined hereinabove, connecting two or more moieties at two or more positions thereof. Preferably, the aryl is phenyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. Substituted heteroaryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroaryl group can be an end group, as this phrase is defined hereinabove, where it is attached to a single adjacent atom, or a linking moiety, as this phrase is defined hereinabove, connecting two or more moieties at two or more positions thereof. Representative examples are pyridine, pyrrole, oxazole, indole, purine and the like.

The term “alkaryl” describes an alkyl, as defined herein, which is substituted by one or more aryl or heteroaryl groups. An example of alkaryl is benzyl.

The term “amine-oxide” describes a —N(OR′) (R″) or a —N(OR′)-group, where R′ and R″ are as defined herein. This term refers to a —N(OR′) (R″) group in cases where the amine-oxide is an end group, as this phrase is defined hereinabove, and to a —N(OR′)-group in cases where the amine-oxime is an end group, as this phrase is defined hereinabove.

As used herein, the term “acyl” refers to a group having the general formula —C(═O)R′, —C(═O)OR′, —C(═O)—O—C(═O)R′, —C(═O)SR′, —C(═O) N(R′)₂, —C(═S)R′, —C(═S)N(R′)₂, and —C(═S)S(R′), —C(═NR′)R″, —C(═NR′)OR″, —C(═NR′)SR″, and —C(═NR′)N(R″)₂, wherein R′ and R″ are each independently hydrogen, halo, substituted or unsubstituted hydroxyl, substituted or unsubstituted thiol, substituted or unsubstituted amine, substituted or unsubstituted acyl, cyclic or acyclic, substituted or unsubstituted, branched or unbranched aliphatic, cyclic or acyclic, substituted or unsubstituted, branched or unbranched heteroaliphatic, cyclic or acyclic, substituted or unsubstituted, branched or unbranched alkyl, cyclic or acyclic, substituted or unsubstituted, branched or unbranched alkenyl, substituted or unsubstituted alkynyl, substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thioxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

As used herein, the term “aliphatic” or “aliphatic group” denotes an optionally substituted hydrocarbon moiety that may be straight-chain (i.e., unbranched), branched, or cyclic (“carbocyclic”) and may be completely saturated or may contain one or more units of unsaturation, but which is not aromatic. Unless otherwise specified, aliphatic groups contain 1-12 carbon atoms. In some embodiments, aliphatic groups contain 1-6 carbon atoms. In some embodiments, aliphatic groups contain 1-4 carbon atoms, and in yet other embodiments aliphatic groups contain 1-3 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

As used herein, the terms “heteroaliphatic” or “heteroaliphatic group”, denote an optionally substituted hydrocarbon moiety having, in addition to carbon atoms, from one to five heteroatoms, that may be straight-chain (i.e., unbranched), branched, or cyclic (“heterocyclic”) and may be completely saturated or may contain one or more units of unsaturation, but which is not aromatic. Unless otherwise specified, heteroaliphatic groups contain 1-6 carbon atoms wherein 1-3 carbon atoms are optionally and independently replaced with heteroatoms selected from oxygen, nitrogen and sulfur. In some embodiments, heteroaliphatic groups contain 1-4 carbon atoms, wherein 1-2 carbon atoms are optionally and independently replaced with heteroatoms selected from oxygen, nitrogen and sulfur. In yet other embodiments, heteroaliphatic groups contain 1-3 carbon atoms, wherein 1 carbon atom is optionally and independently replaced with a heteroatom selected from oxygen, nitrogen and sulfur. Suitable heteroaliphatic groups include, but are not limited to, linear or branched, heteroalkyl, heteroalkenyl, and heteroalkynyl groups.

The term “halo” describes fluorine, chlorine, bromine or iodine substituent.

The term “halide” describes an anion of a halogen atom, namely F⁻, Cl⁻ Br⁻ and I⁻.

The term “haloalkyl” describes an alkyl group as defined above, further substituted by one or more halide.

The term “sulfate” describes a —O—S(═O)₂—OR′ end group, as this term is defined hereinabove, or an —O—S(═O)₂—O— linking moiety, as these phrases are defined hereinabove, where R′ is as defined hereinabove.

The term “thiosulfate” describes a —O—S(═S)(═O)—OR′ end group or a —O—S(═S)(═O)—O— linking moiety, as these phrases are defined hereinabove, where R′ is as defined hereinabove.

The term “sulfite” describes an —O—S(═O)—O—R′ end group or a —O—S(═O)—O— group linking moiety, as these phrases are defined hereinabove, where R′ is as defined hereinabove.

The term “thiosulfite” describes a —O—S(═S)—O—R′ end group or an —O—S(═S)—O— group linking moiety, as these phrases are defined hereinabove, where R′ is as defined hereinabove.

The term “sulfinate” or “sulfinyl” describes a —S(═O)—OR′ end group or an —S(═O)—O—group linking moiety, as these phrases are defined hereinabove, where R′ is as defined hereinabove.

The terms “sulfoxide” or “sulfinyl” describe a —S(═O) R′ end group or an —S(═O)— linking moiety, as these phrases are defined hereinabove, where R′ is as defined hereinabove.

The term “sulfonate” or “sulfonyl” describes a —S(═O)₂—R′ end group or an —S(═O)₂— linking moiety, as these phrases are defined hereinabove, where R′ is as defined herein.

The term “S-sulfonamide” describes a —S(═O)₂—NR′R″ end group or a —S(═O)₂—NR′— linking moiety, as these phrases are defined hereinabove, with R′ and R″ as defined herein.

The term “N-sulfonamide” describes an R′S(═O)₂—NR″— end group or a —S(═O)₂—NR′— linking moiety, as these phrases are defined hereinabove, where R′ and R″ are as defined herein.

The term “disulfide” refers to a —S—SR′ end group or a —S—S— linking moiety, as these phrases are defined hereinabove, where R′ is as defined herein.

The term “phosphate” describes an —O—P(═O)₂(OR′) end or reactive group or a —O—P(═O)₂(O)— linking moiety, as these phrases are defined hereinabove, with R′ as defined herein.

The term “phosphonate” describes a —P(═O)(OR′)(OR″) end or reactive group or a —P(═O)(OR′)(O)— linking moiety, as these phrases are defined hereinabove, with R′ and R″ as defined herein.

The term “thiophosphonate” describes a —P(═S)(OR′)(OR″) end group or a —P(═S)(OR′)(O)— linking moiety, as these phrases are defined hereinabove, with R′ and R″ as defined herein.

The term “carbonyl” or “carbonate” as used herein, describes a —C(═O)—R′ end group or a —C(═O)— linking moiety, as these phrases are defined hereinabove, with R′ as defined herein.

The term “thiocarbonyl” as used herein, describes a —C(═S)—R′ end group or a —C(═S)— linking moiety, as these phrases are defined hereinabove, with R′ as defined herein.

The term “oxo” as used herein, described a ═O end group.

The term “thioxo” as used herein, described a ═S end group.

The term “oxime” describes a ═N—OH end group or a ═N—O— linking moiety, as these phrases are defined hereinabove.

The term “hydroxyl” describes a —OH group.

As used herein, the term “aldehyde” refers to an —C(═O)—H group.

The term “acyl halide” describes a —(C═O)R″″ group wherein R″ is halo, as defined hereinabove.

The term “alkoxy” as used herein describes an —O-alkyl, an —O-cycloalkyl, as defined hereinabove. The ether group —O— is also a possible linking moiety.

The term “aryloxy” describes both an —O-aryl and an —O-heteroaryl group, as defined herein.

The term “disulfide” as used herein describes an —S—S— linking moiety, which in some cases forms between two thiohydroxyl groups.

The terms “thio”, “sulfhydryl” or “thiohydroxyl” as used herein describe an —SH group.

The term “thioalkoxy” or “thioether” describes both a —S-alkyl group, and a —S-cycloalkyl group, as defined herein. The thioether group —S— is also a possible linking moiety.

The term “thioaryloxy” describes both a —S-aryl and a —S-heteroaryl group, as defined herein. The thioarylether group —S-aryl- is also a possible linking moiety.

The term “cyano” or “nitrile” describes a —C≡N group.

The term “isocyanate” describes an —N═C═O group.

The term “nitro” describes an —NO₂ group.

The term “carboxylate” or “ester”, as used herein encompasses C-carboxylate and O-carboxylate.

The term “C-carboxylate” describes a —C(═O)—OR′ end group or a —C(═O)—O— linking moiety, as these phrases are defined hereinabove, where R′ is as defined herein.

The term “O-carboxylate” describes a —OC(═O) R′ end group or a —OC(═O)— linking moiety, as these phrases are defined hereinabove, where R′ is as defined herein.

The term “thiocarboxylate” as used herein encompasses “C-thiocarboxylate and O-thiocarboxylate.

The term “C-thiocarboxylate” describes a —C(═S)—OR′ end group or a —C(═S)—O— linking moiety, as these phrases are defined hereinabove, where R′ is as defined herein.

The term “O-thiocarboxylate” describes a —OC(═S) R′ end group or a —OC(═S)— linking moiety, as these phrases are defined hereinabove, where R′ is as defined herein.

The term “carbamate” as used herein encompasses N-carbamate and O-carbamate.

The term “N-carbamate” describes an R″OC(═O)—NR′— end group or a —OC(═O)—NR′—linking moiety, as these phrases are defined hereinabove, with R′ and R″ as defined herein.

The term “O-carbamate” describes an —OC(═O)—NR′R″ end group or an —OC(═O)-NR′— linking moiety, as these phrases are defined hereinabove, with R′ and R″ as defined herein.

The term “thiocarbamate” as used herein encompasses N-thiocarbamate and O-thiocarbamate.

The term “O-thiocarbamate” describes a —OC(═S)—NR′R″ end group or a —OC(═S)—NR′— linking moiety, as these phrases are defined hereinabove, with R′ and R″ as defined herein.

The term “N-thiocarbamate” describes an R″OC(═S)NR′— end group or a —OC(═S)NR′— linking moiety, as these phrases are defined hereinabove, with R′ and R″ as defined herein.

The term “dithiocarbamate” as used herein encompasses N-dithiocarbamate and S-dithiocarbamate.

The term “S-dithiocarbamate” describes a —SC(═S)—NR′R″ end group or a —SC(═S)NR′— linking moiety, as these phrases are defined hereinabove, with R′ and R″ as defined herein.

The term “N-dithiocarbamate” describes an R″SC(═S)NR′— end group or a —SC(═S)NR′— linking moiety, as these phrases are defined hereinabove, with R′ and R″ as defined herein.

The term “urea”, which is also referred to herein as “ureido”, describes a —NR′C(═O)—NR″R′″ end group or a —NR′C(═O)—NR″— linking moiety, as these phrases are defined hereinabove, where R′ and R″ are as defined herein and R″ is as defined herein for R′ and R″.

The term “thiourea”, which is also referred to herein as “thioureido”, describes a —NR′—C(═S)—NR″R′″ end group or a —NR′—C(═S)—NR″— linking moiety, with R′, R″ and R′″ as defined herein.

The term “amide” as used herein encompasses C-amide and N-amide.

The term “C-amide” describes a —C(═O)—NR′R″ end group or a —C(═O)—NR′— linking moiety, as these phrases are defined hereinabove, where R′ and R″ are as defined herein.

The term “N-amide” describes a R′C(═O)—NR″— end group or a R′C(═O)—N— linking moiety, as these phrases are defined hereinabove, where R′ and R″ are as defined herein.

The term “imine”, which is also referred to in the art interchangeably as “Schiff-base”, describes a —N═CR′— linking moiety, with R′ as defined herein or hydrogen. As is well known in the art, Schiff bases are typically formed by reacting an aldehyde or a ketone and an amine-containing moiety such as amine, hydrazine, hydrazide and the like, as these terms are defined herein. The term “aldimine” refers to a —CH═N— imine which is derived from an aldehyde. The term “ketimine” refers to a —CR′═N— imine which is derived from a ketone.

The term “hydrazone” refers to a —R′C═N—NR″— linking moiety, wherein R′ and R″ are as defined herein.

The term “semicarbazone” refers to a linking moiety which forms in a condensation reaction between an aldehyde or ketone and semicarbazide. A semicarbazone linking moiety stemming from a ketone is a —R′C═NNR″C(═O)NR′″—, and a linking moiety stemming from an aldehyde is a —CR′═NNR″C(═O)NR′″—, wherein R′ and R″ are as defined herein and R″ or as defined for R′.

As used herein, the term “lactone” refers to a cyclic ester, namely the intra-condensation product of an alcohol group-OH and a carboxylic acid group-COOH in the same molecule.

As used herein, the term “lactam” refers to a cyclic amide, as this term is defined herein. A lactam with two carbon atoms beside the carbonyl and four ring atoms in total is referred to as a β-lactam, a lactam with three carbon atoms beside the carbonyl and five ring atoms in total is referred to as a γ-lactam, a lactam with four carbon atoms beside the carbonyl and six ring atoms in total is referred to as a 8-lactam, and so on.

The term “guanyl” describes a R′R″NC(═N)— end group or a —R′NC(═N)— linking moiety, as these phrases are defined hereinabove, where R′ and R″ are as defined herein.

The term “guanidine” describes a —R′NC(═N)—NR″R′″ end group or a —R′NC(═N)—NR″— linking moiety, as these phrases are defined hereinabove, where R′, R″ and R″ are as defined herein.

The term “hydrazine” describes a —NR′—NR″R′″ end group or a —NR′—NR″— linking moiety, as these phrases are defined hereinabove, with R′, R″, and R″ as defined herein.

As used herein, the term “hydrazide” describes a —C(═O)—NR′—NR″R′″ end group or a —C(═O)—NR′—NR″— linking moiety, as these phrases are defined hereinabove, where R′, R″ and R′″ are as defined herein.

The term “hydroxylamine”, as used herein, refers to either a —NHOH group or a —ONH₂.

As used herein, the terms “azo” or “diazo” describe a —N═N—R′ end group or a —N═N— linking moiety, as these phrases are defined hereinabove, where R′ is as defined herein.

As used herein, the term “azido” described a —N═N⁺═N⁻(—N₃) end group.

The term “triazine” refers to a heterocyclic ring, analogous to the six-membered benzene ring but with three carbons replaced by nitrogen atoms. The three isomers of triazine are distinguished from each other by the positions of their nitrogen atoms, and are referred to as 1,2,3-triazine, 1,2,4-triazine, and 1,3,5-triazine. Other aromatic nitrogen heterocycles include pyridines with 1 ring nitrogen atom, diazines with 2 nitrogen atoms in the ring and tetrazines with 4 ring nitrogen atoms.

The term “triazole” refers to either one of a pair of isomeric chemical compounds with molecular formula C₂H₃N₃, having a five-membered ring of two carbon atoms and three nitrogen atoms, namely 1,2,3-triazoles and 1,2,4-triazoles.

The term “aziridine”, as used herein, refers to a reactive group which is a three membered heterocycle with one amine group and two methylene groups, having a molecular formula of —C₂H₃NH.

As used herein, the term “thiohydrazide” describes a —C(═S)—NR′—NR″R′″ end group or a —C(═S)—NR′—NR″— linking moiety, as these phrases are defined hereinabove, where R′, R″ and R″ are as defined herein.

As used herein, the term “methyleneamine” describes an —NR′—CH₂—CH═CR″R′″ end group or a —NR′-CH₂—CH═CR″— linking moiety, as these phrases are defined hereinabove, where R′, R″ and R′″ are as defined herein.

The term “diene”, as used herein, refers to a —CR′═CR″—CR′″═CR″— group, wherein R′ as defined hereinabove, and R″, R″ and R″″ are as defined for R′.

The term “dienophile”, as used herein, refers to a reactive group that reacts with a diene, typically in a Diels-Alder reaction mechanism, hence a dienophile is typically a double bond or an alkenyl.

The term “epoxy”, as used herein, refers to a reactive group which is a three membered heterocycle with one oxygen and two methylene groups, having a molecular formula of —C₂H₃O.

The phrase “covalent bond”, as used herein, refers to one or more pairs of electrons that are shared between atoms in a form of chemical bonding.

It is expected that during the life of a patent maturing from this application many relevant cyclic peptides with affinity to EGFR and mutants thereof will be developed and the scope of the phrase “cyclic peptides” is intended to include all such new technologies a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate some embodiments of the invention in a non-limiting fashion.

### Example 1

### Materials and Methods

### Cells and Culture Conditions:

Lung cancer (H1299, H1975), glioblastoma (DKMG; also known as CVCL_1173 cell line), myeloid leukemia (K562), breast cancer (MDA-435), normal breast (MCF-10A) and embryonic (HEK-293) cell lines were cultured in RPMI 1640 or DMEM medium supplemented with 10% fetal bovine serum (FBS), 2 mM L-glutamine, 1% penicillin/streptomycin (all from Biological Industries, Israel). Cells were maintained at 37° C. in humidified 5% CO₂ atmosphere. H1299, H1975 and DKMG cells are characterized by cell surface overexpression of EGFR. DKMG cells are known to express two forms of EGFR-wild type (WT) and another carrying a mutation in the external region of receptor (EGFRvIII). H1975 cells (also known as NCI-H1975 or CRL-5908) carry a mutation in the internal domain of EGFR (L858R/T790M).

### Positive In Vitro Biopanning Selection for Specific Peptides:

A heptapeptide phage display library (Ph.D.-C7C Phage Display Library Kit, New England Biolabs, USA) was used for the in vitro biopanning experiments. All cell lines were individually incubated with phages from the stock library. K562 cells, which do not express EGFR were used for negative biopanning selection. Adherent cells (H1299, H1297 and DKMG) were plated at a density of 10⁵ cells/ml in a 6-well plate. When the culture reached 80% confluence, the first well was incubated with phage library (10¹¹ pfu/10 μl) for 1 h at 37° C. with gentle stirring. The medium containing unbound phage was collected and transferred to the second, similar well and incubated again. The procedure was repeated with the third well. Cells in the third wells were washed four times with 0.5% Tween-20 in PBS. Then, 0.5 ml of elution buffer (0.2 M glycine-HCl, pH 2.2) was added and incubated for 10 min at 4° C.; the pH was neutralized by addition of 75 μl of 1 M Tris-HCl buffer, pH 9.0. The supernatant containing cell surface binding phage was collected. The cells were then lysed by incubation for 1 h at 4° C. with 2 ml of 30 mM Tris-HCl and 1 mM EDTA, pH 8.0. The medium was collected and centrifuged at 1,500 rpm for 5 min and the supernatant containing cell-internalized phage was transferred to a new tube. The internalized and surface-bound phages were amplified according to the manufacturer's instructions. An aliquot of the phages was retained for sequencing (see below) and the remainder was subjected to two additional rounds of biopanning. For K562 suspension cells, the same scheme was used, except that the cells were washed by centrifugal pelleting for 5 min, RT, 1,800 rpm.

### DNA Preparation and Sequencing:

Following biopanning, DNA was extracted from first and third round phage as well as from the original phage pool according to the manufacturer's instructions. Libraries were prepared for NGS, and sequencing was performed by Hylabs Pty Ltd. using MiSeq technology (Rehovot, Israel).

### Analysis of NGS Data:

For the analysis of DNA sequencing data, a specific script was written. The workflow of the data processing included translation; generation of a report list sorted by the number of amino acid sequence repeats; curation of the list by removal of sequences internalized by K562 cells or those that did not appear in the original Ph.D. C7C original phage display library. From this workflow, 11 peptides were selected from which two sub-lists were generated: one—a list of peptides internalized by all 3 cell lines H1299, H1975, DKMG; and second—a list of peptides internalized uniquely by each cell line.

### Solid Phase Synthesis of Peptides:

Eleven 7-mer S—S bridged cyclic peptides (total of 9 amino acid in each sequence including terminal cysteines) were synthetized by solid phase peptide synthesis on Rink Amide resin (loading 0.4-0.8 mmol/g, 100-200 mesh, Sigma-Aldrich) either as free peptides or conjugated to fluorescein 5-isothiocyanate (FITC) or camptothecin (CPT; (S)-(+)-camptothecin) through y-aminobutyric acid GABA linker using standard Fmoc protocols. The cyclization between two cysteine amino acids of the peptides was carried out prior to conjugation step using I₂ (10 eq.) in DMF/H₂O (4:1) for 2 hours.

### Peptide Conjugation to FITC and CPT:

The Fmoc-deprotected cyclic peptides on the resin were coupled to an Fmoc-GABA-OH linker (2 eq.) using PyBop (2 eq.) as a coupling reagent and DIPEA (6 eq.) as a base in DMF during 2 hours. Fmoc was removed (20% piperidine in DMF 2×15 min), resin was washed (DMF 3×3 min, DCM 3×3 min) and the deprotected peptidyl resin was reacted as a follows:

For FITC conjugation: FITC (2 eq.) in DMF in the presence of DIPEA (6 eq.) during 2 hours;

For CPT conjugation: (S)-4-ethyl-3,14-dioxo-3,4,12,14-tetrahydro-1H-pyrano[3′,4′: 6,7]indolizino[1,2-b]quinolin-4-yl (4-nitrophenyl) carbonate (CPT-OPNF) (2 eq.) in DMF in the presence of DIPEA (6 eq.) and DIMAP (0.5 eq.), during 2 hours.

The resin was washed (DMF 3×3 min, DCM 3×3 min), dried and cleaved by treating with cold TFA cocktail (95% TFA, 2.5% TIS, 2.5% H₂O) for 2 hours. Further, the solution of PDC's was evaporated (N₂) and the crude was purified by preparative HPLC (ECOM preparative system, with dual UV detection; column: Phenomenex Gemini® 10 μm RP18 110 Å, LC 250×21.2 mm, 25° C.; eluent A (0.1% FA in water) and B (CH₃CN) were used. A typical elution was a gradient from 100% A to 100% B over 35 min at a flow rate of 25 ml/min.

For the P6-FITC conjugate: (42% yield, purity 95%) LC-MS: RT=8.66 min; MS: (ESI-MS) m/z calcd: 1448.5, found: 725.26 [M+2H]²⁺. For the P6-CPT conjugate: (50% yield, purity 27%) LC-MS: RT=4.79 min; MS: (ESI-MS) m/z calcd: 1433.55, found: 717.9 [M+2H]²⁺. For the P9-CPT conjugate: (45% yield, purity 60%) LC-MS: RT=5.21 min; MS: (ESI-MS) m/z calcd: 1473.52 found: 737.8 [M+2H]²⁺.

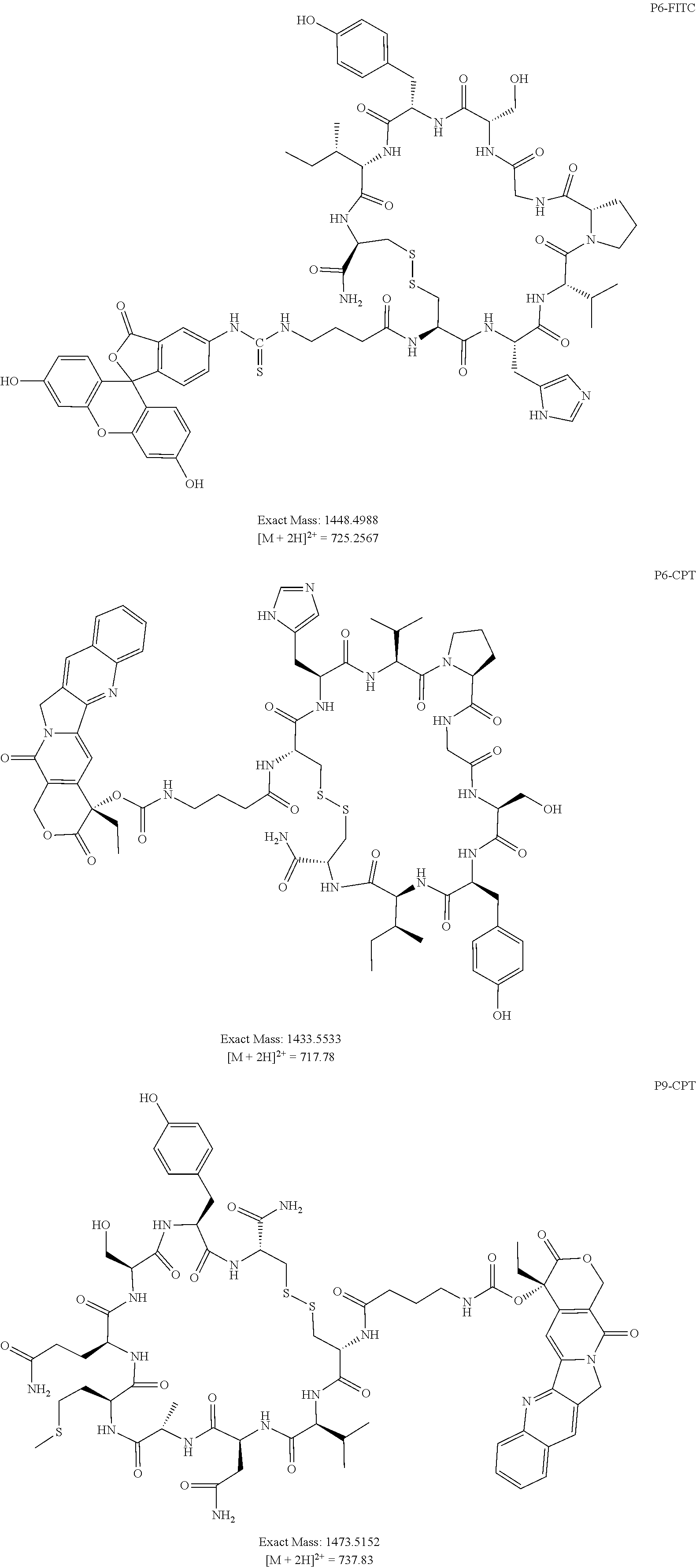

### Peptide Drug Conjugate Stability Study:

Peptide-FITC and peptide-CPT conjugates were incubated at 37° C. in 1 ml of DMEM or RPMI full growth medium for 0, 0.6, 3, 7, 24, 48, and 72 h. At each time point, 10 μl of sample was taken and mixed with 25 μl ACN/H₂O (1:1, v.v.). The samples were centrifuged at 1,400 rpm for 15 min. Supernatants were collected, filtered and analyzed by liquid chromatography mass spectrometry (LC-MS).

### Detection of EGFR+Ve/EGFRvIII Expression by Flow Cytometry:

H1299 and DKMG adherent cells were collected after treatment with trypsin, resuspended in Flow Cytometry Staining Buffer (FCSB, Biological Ind., Israel) and centrifuged for 5 min 1,500 rpm. K562 suspension cells were directly resuspended in FCSB. A population of 10⁶ cells/ml were then incubated with anti-human EGFR-APC (3 μl) antibodies at 4° C. for 1 h in the dark, or mouse anti-human EGFRvIII primary (3 μl) followed by two washings with FCSB. Then, 3 μl of APC labeled with either anti-mouse Ig antibody were added and the cells incubated at 4° C. for 20 min in the dark. After washing, cells were resuspended in 200 μl of FCSB and analyzed with Cytoflex, Beckmann coulter cell analyzer. For each sample, 5×10⁴ cells were tested. FlowJo software was used to analyze the data.

### Binding Analysis of Peptide-FITC Conjugates:

Cells with 80-90% confluence were incubated with 2.5 μM FITC-conjugated peptides in RPMI-5% FBS for 1 h at 37° C. Cells were scrapped from the culture flask and washed twice with 2 ml FCSB. Cells were analyzed as previously described Internalization of peptide-FITC conjugates.

Cells were cultured overnight in 6-well plates under 80-90% confluence, the medium was replaced with 1 ml of the fresh medium with 5% FBS that contained 2.5 μM FITC-conjugated peptides. The plates were incubated for 1, 2, or 3 h at 37° C. After incubation, the cells were collected with trypsin, washed twice with FCSB, and resuspended in 200 μl of FCSB. Analysis was done with FACS cell analyzer.

### Confocal Microscopy:

H1299, DKMG and HEK-293 cells were seeded in 12-well glass bottom black plates in concentrations of 5.0×10⁴, 1.0×10⁵, and 2.5×10⁴ cells/well, respectively. The cells were cultured in complete growth medium at 37° C. under 5% CO₂ for 24 h. After incubation, the medium was removed, the cells were twice washed with PBS Ca²⁺Mg²⁺, and new fresh medium (0.5 ml) contained 25 μM of FITC labeled P6 or P9 peptides was added. The stained cells were incubated at 37° C. under 5% CO₂ for 0 h and 3 h, washed twice with PBS, and stained with Hoechst 33258 (16.23 mM, 0.5 ml, 37° C., 5% CO₂) for 10 min. The cells were twice washed with PBS and fixed with 4% paraformaldehyde (PFA). Fluorescence images were then recorded at the magnification of ×200 with a Zeiss LSM700 confocal laser microscope.

### Competitive Binding Assay:

H1299 and DKMG cell lines were cultured in complete RPMI growth medium in 6 well plate overnight, until 80% confluence. Pre-incubation was performed with blocking buffer consisting of 5 mg/ml BSA in PBS, pH 7.4 at 4° C. for 30 minutes. Then, cells were washed twice in PBS and incubated with 2.5 μM of FITC-labeled peptides with or without 80 nM EGF at 4° C. for 1 hour. The cells were then incubated for 15 min at 37° C. under 5% CO₂ to allow for peptide internalization. The cells were washed twice by centrifugation at 1,500 rpm for 5 min and analyzed by flow cytometry.

### Peptide Docking:

The three-dimensional structures of the peptides were predicted using the PEPstrMOD server. The EGFR structure (Protein Data Bank ID 1IVO) was used for docking simulations of the peptides to the receptor. This structure represents the human EGFR extracellular region in complex with EGF ligands. The ligands were removed from the structures prior to the docking simulations. Unbiased rigid body docking (exhaustive search of all possible binding sites and binding poses) was done using three servers, HDOCK, LZerD, and ZDOCK. Next, the best binding poses obtained from each of the three servers were optimized using local docking protocol of the RosettaDock server that identifies low-energy conformations by optimizing rigid-body orientations and side-chain conformations. The best three local docking poses of each optimization run (total of nine) were minimized using UCSF Chimera and the peptide-EGFR binding energy was assessed using PRODIGY.

### Cellular Toxicity of the Peptide-CPT Conjugates:

Cell growth in the presence of PDCs was measured by a commercial XTT assay kit (Cell Proliferation Kit, XTT based; Biological Industries, Israel). The cells (10⁴ cells/well) were seeded in 96 well plates and incubated overnight in complete growth medium. The cells were washed and then cultured in 100 μl of new medium containing various concentrations of drug or peptides, for 24 h, 48 h, and 72 h. The XTT solution was added to each well and the plate was further incubated 2-3 h at 37° C. The optical density in the wells was measured at both 480 nm and 680 nm using a TECAN Infinite M200 ELISA reader. All the tests were done in triplicates, and each experiment was repeated three times.

### In Vivo Targeting:

Animal protocols were approved by the Institutional Animal Care and Use Committee at Ariel university Israel. Nude mice were injected with either H1299 cells in the right flank and with K562 cells in the left flank, so that each mouse bore either dual H1299 and K562 xenografts. When tumors reached, 100 mm³ mice were injected via tail vein with 100 μg of FITC-labeled cyclic peptide, according to some embodiments of the present invention, and imaged using IVIS® SpectrumCT 8- and 24-hours post injection. Immediately afterwards, the mice were sacrificed for ex vivo imaging of organs.

### Statistical Analysis:

All experiments were conducted in triplicate and the results expressed as the mean±standard deviation. Data were analyzed using GraphPad Prism 6.0 (GraphPad Software, Inc. CA, USA). Statistical analysis was performed by two-way ANOVA test. P-value<0.05 was considered to indicate a statistically significant difference.

### Example 2

### Results

### Evaluation of Cell Surface Expression of EGFRWT and EGFRvIII by Flow Cytometry:

Prior to using H1299, H1975, DKMG, and K562 cells in biopanning, the expression of EGFRWT and EGFRvIII was confirmed using flow cytometry analysis. As expected, flow cytometry analysis showed that H1299, H1975, and DKMG expressed EGFRWT (65%, 55%, and 92%, respectively), whereas only DKMG cells expressed EGFRvIII (82%). K562 cells did not express either of these receptors and were used as negative control.

### Selection of EGFR-Specific Peptides:

Peptides that specifically bind and internalize into EGFR⁺ve cells were isolated using the Ph.D-C7C peptide phage library. For each cell line, 3 rounds of biopanning were performed and both surface and internalized phages were recovered. DNA from phage pools were extracted from the first and the third rounds sequenced by NGS and translated. To select for target-specific peptides, the inventors first validated that all the peptide sequences appeared in the original phage display library. Then, peptides derived from the EGFR⁻ve K562 cells were excluded from the list. After this step, 617,503 sequences remained.

Finally, two groups of peptides were constructed; the first included sequences that were present in all three EGFR+ve cell lines and included about 416 sequences; the second was divided into three subgroups, one for each cell line, wherein the subgroup for H1299 included some 1666 sequences, the subgroup for H1975 included some 10437 sequences, the subgroup for DKMG included some 981 sequences. From this list, top eleven S—S bridged cyclic peptides sequences were selected by their count in the sequencing data (the highest number of reads).

For the sequence analysis, a script was developed for data processing, which included:
1. Translation of each three-base codon into an amino acid sequence from DNA sequence to the protein level.
2. Counting the number of times each sequence (peptide) appeared in the sequence file;
3. Generating a report list sorted by the number of the appearance and then alphabetically;
4. Exclusion of peptides that did not appear in the original phage display library kit (for example, the Ph.D.-C7C Phage Display Library Kit #E8120, by New England Biolabs INC, USA.); The heptapeptide phage display library which is a combinatorial library of random peptides with a disulfide constrained loop in contrast to the Ph.D.™-12 Phage Display Peptide Library and Ph.D.™-7 Phage Display Peptide Library which are a linear peptide. This exclusion step was used as positive control to filter out sequences that were created by a sequencing error and do not appear in the original library;
5. Exclusion of peptides that appeared in the samples from the EGFR negative cells (K562 cell line); This exclusion step was used as negative control to filter out sequences that bind and internalized to the EGFR−ve cells and indicate nonspecific binding.

This data processing led to the creation of two groups of peptides, the first one included sequence that were present in all three cell lines of the interest and the second one consisted of three subgroups, each of them included unique sequences for each cell line.

### Binding and Internalization of the Selected Peptides:

The binding and internalization capabilities of the cyclic peptides provided herein have been assayed using fluorescein 5-isothiocyanate (FITC; a fluorescence probe for labeling amines) as a detectable moiety (label). Eleven selected FITC-labeled cyclic peptides, P1-P11 (2.5 μM of each) were tested on K562, H1299, and DKMG cells, as described above. This concentration was chosen based on preliminary dose dependence study. The total number of cells that bound but did not internalize peptide was calculated by subtracting the number of peptide internalized cells from the total number of the positive cells.

Flow cytometry analysis showed that FITC-labeled peptides P2-P6, P8, P9, and P11 peptides bind to H1299 and DKMG selectively but not to K562 cells. Furthermore, the FITC-labeled P3, P6, P8, P9, and P11 peptides internalized only into to EGFR+ve cells. FITC-labeled P3-P5, and P9 showed higher internalization activity to DKMG cells line expressing both EGFR+ve and EGFRvIII mutation. Table 3 presents the peptide specificity per cell line (the table refers to the source of the cells from which the peptides were initially identified).

TABLE 3

| | | SEQ | |
|---|---|---|---|
| | | ID | |
| DENOTATION | SEQUENCE | No. | Cell line |
| P1 | CLRWRFGRC | 1 | H1299, DKMG, |
| | | | H1975 |
| P2 | CSAETVESC | 2 | H1299, DKMG, |
| | | | H1975 |
| P3 | CVRWRFGRC | 3 | H1299, DKMG, |
| | | | H1975 |
| P4 | CLAVEVRPC | 4 | H1299, DKMG, |
| | | | H1975 |
| P5 | CPNDSYHQC | 5 | H1299 |
| P6 | CHVPGSYIC | 6 | H1299 |
| P7 | CWHSLSLAC | 7 | H1975 |
| P8 | CSALWASHC | 8 | H1975 |
| P9 | CVNAMQSYC | 9 | DKMG |
| P10 | CNWLSRTEC | 10 | DKMG |
| P11 | CAQYTPGRC | 11 | DKMG |

### Competitive Binding of Peptides to EGFR:

To validate whether the internalized peptide target the EGFR receptor, a competitive binding assay with the natural ligand EGF was conducted. Competitive uptake of labeled cyclic peptides by fluorescent-activated cell sorting (FACS) assay in absence or presence of 80 nM EGF protein in H1299 cells (30,000 cells) showed that EGF successfully competed with the peptides, significantly reducing the binding of the FITC-labeled cyclic peptides.

### Evaluation of Target Specificity of the Peptides Towards EGFR Expressed Cells by Flow Cytometry:

Targeting specificity of FITC-labeled P4-P6, P8, and P9 peptides was investigated on three cell lines, which were not used in biopanning: kidney HEK-293 and breast MCF-10A normal cells, and breast cancer MDA-435 cells. To verify EGFR WT expression on these cell lines, the inventors stained the cells with APC-labeled monoclonal anti-human EGFR antibody followed by a flow cytometry analysis. EGFR WT expression on HEK-293 and MCF-10A cells was found to be negligible (less than 10%), while about 97% of MDA-435 cells were EGFR WT positive.

The interaction of the FITC labeled P4 P6, P8, and P9 with these cell lines was analyzed by flow cytometry. All the investigated cyclic peptides were found to have low binding capacity to EGFR-negative HEK-293 cells, while P6 and P9 showed a pronounced internalization into high-EGFR-expressing MDA-435 cells; P8 was less specific exhibiting a noticeable binding to both MCF-10A and MDA-435 cell lines.

### Evaluation of Target Specificity of the Peptides Towards EGFR Expressed Cells by Fluorescence Microscopy:

To get additional confirmation data on the specificity of the FITC labeled P6 and P9 peptides towards EGFR H1299 lung cancer and EGFRVIII DKMG glioblastoma cell lines compared to EGFR−ve HEK-293 normal cells, the inventors employed confocal scanning microscopy. The cells were incubated with 25 μM FITC-labeled P6 and P9, the cell nuclear were stained with Hoechst-293, the cells were fixed with 4% paraformaldehyde, and captured immediately after addition and 3 h later. Notably, all the cells exhibited blue fluorescence signal of Hoechst-293, which evidenced their viability. A clear correlation of the green FITC signal with the cell type was established. After 3 hours, a strong signal was observed from the FITC-P6-stained H1299 and DKMG cells expressing EGFR, but low-intensive signal from HEK-293 cells lacking these receptors. In contrast, FITC-P9 exhibited a high binding affinity and specificity to DKMG (strong green signal). While the fluorescence intensity from FITC-P6 on H1299 and DKMG was not detectable at time zero, FITC-P9 demonstrated a pronounced emission on DKMG, which significantly increased after 3 hours.

### Peptide Docking:

Unbiased rigid body docking simulations were carried out between the cyclic peptides and the EGFR receptor to identify their binding sites within the receptor. The docking simulation showed that most of the docked structures bind to the receptor in the cavity between domain I and III. Similar results were obtained with the LZerD and ZDOCK docking servers. After identification of the binding site, optimization of the binding pose was carried out using the RosettaDock server. The best docked pose of each peptide from each server undergoes local docking protocol that enables side chain movements. The best three obtained locally docked structures (of each peptide) were then minimized and the binding energy of the peptide to the receptor was assessed using PRODIGY. According to the docking results, the peptides are bound to the EGFR receptor in vicinity to the EGF binding site. P6 and P9 show substantial overlap with EGF while P11 is bound deeper within the cavity (toward domain II) and have a smaller overlap with EGF. The PROGIDY binding energies are −13.0, −11.9, and −11.3 kcal mol⁻¹ for peptides P6, P9, and P11, respectively.

### Synthesis of Peptide CPT Conjugates:

Taking together all the previous results, the most promising peptides, as defined by the broadest binding and internalization across the panel of cell lines and by their competitive ability and docking, were chosen for further study. The two most promising peptide candidates P6 and P9 were conjugated with Camptothecin (CTP). CPT, a kind of alkaloid, is a DNA topoisomerase I (Topo I) inhibitor with an IC₅₀ of 679 nM. CPT exhibits powerful antineoplastic activity against colorectal, breast, lung and ovarian cancers, modulates hypoxia-inducible factor-1α (HIF-1α) activity by changing microRNAs (miRNA) expression patterns in human cancer cells.

First, on-resin intermediates P6 and P9 with deprotected GABA N-terminus were obtained after Fmoc removal of their precursors (20% piperidine in NMP, 10 mL). Notably, in its free form, CPT in particular is very potent, but it has poor solubility and produces off-target cytotoxicity, factors that have precluded its clinical development. The inventors hypothesized that in general, conjugation of CPT to a targeting peptide would improve its pharmacological properties. Thus, the free N-terminus amine of the resulting P6 and P9 were loaded with activated CPT leading to creation of biodegradable carbamate linkage. Finally, all the on-resin synthesized 10 conjugates were cleaved from the solid support and precipitated by the addition of cold diethyl ether, isolated, lyophilized, and identified by LC-MS and HRMS, as peptide conjugates P6-CTP and P9-CPT. Scheme 1 below presents the synthesis of the P6-CTP and P9-CPT conjugates.

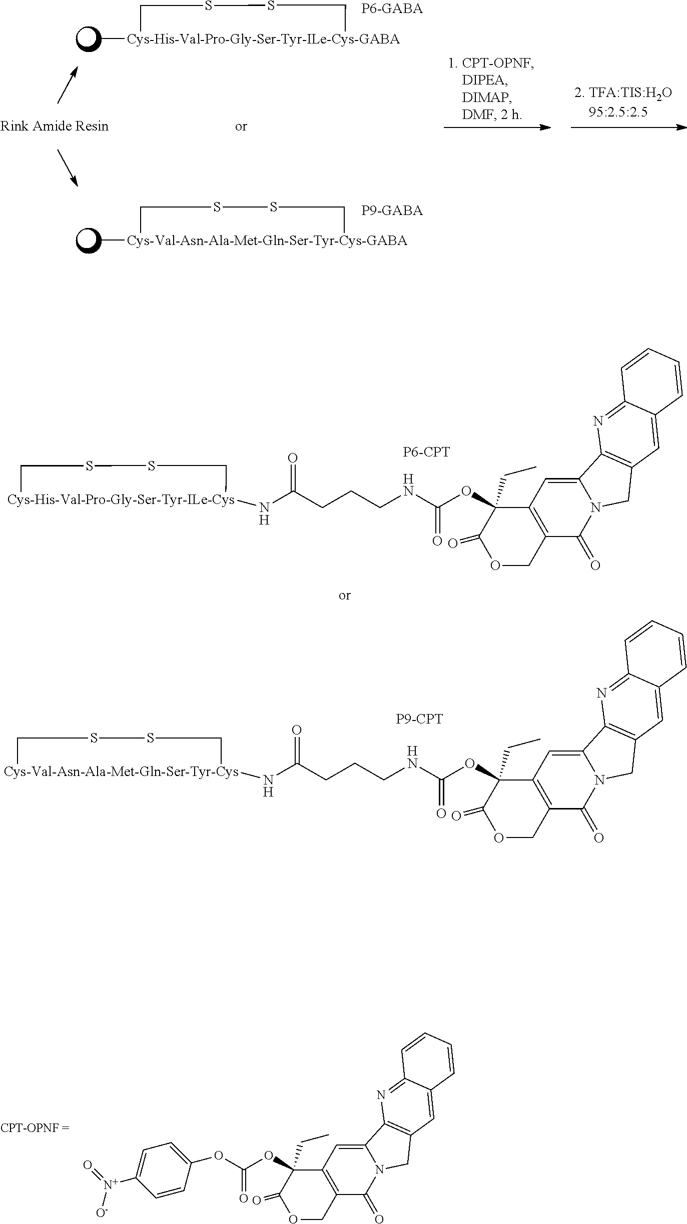

### Stability of Peptide-Drug Conjugates:

The stability of P6-CPT and P9-CPT was measured in RPMI and DMEM complete growth medium at 37° C. for 0 h, 0.5 h, 3 h, 7 h, 24 h, 48, and 72 h. The degradation of the conjugates and the CPT release was analyzed by LC-MS. The stability of these conjugates and drug release rates were similar. The conjugates degraded with a half-life of 6-7 hours.

Nevertheless, the stabilities of both conjugates were potentially sufficient to provide potent anticancer activities. Therefore, next the cytotoxicity of these conjugates was tested in cancer cell lines.

### Effect of P6-CPT and P9-CPT on Cell Viability:

The cytotoxicity of the Peptide-Drug conjugates (PDCs) was tested in a range of concentrations (0.5-50 μM) against H1299 and DKMG cells using two protocols. In the first protocol, cells were pre-incubated with P6, P9, P6-CPT, and P9-CPT for 6 hours, the medium was removed, and a fresh, drug-free complete medium was added for additional 24 h (designated as 6h24h) or 48 h (6h48h). In the second protocol, cells were exposed continuously to PDCs for 24 h, 48 h, and 72 hours. The IC₅₀ were calculated, and results are presented in Table 4 and described below.

H1299: According to the first protocol, P6 and P9 did not demonstrate cytotoxicity on the H1299 cells even after 48 h of preincubation while P6-CPT and P9-CPT exhibited cytotoxic effect after 48 h though the IC₅₀ was higher than cytotoxic effects of CPT alone (see, Table 4). However, in the second protocol with continuous exposure cytotoxicity of P6-CPT and P9-CPT was observed after 24 hours post exposure. The IC₅₀ of these PDCs was 2-3-fold lower compared to free CPT (Table 4).

DKMG: After 24 h of pre-incubation with P9 and P9-CPT, according to the first protocol, the DKMG cell viability was not affected. The cytotoxic effect of P9-CPT on DKMG was noted after 48 hours while its IC₅₀ concentration was 4-fold higher compared to the free CPT's alone (Table 4). After the continuous incubation during 24 h (the second protocol), P9-CPT conjugate was observed to be more toxic (IC₅₀=6.76 μM) compared to the free CPT (IC₅₀=25 μM). After 48 h and 72 h of incubation (the second protocol), P9-CPT demonstrated a similar cytotoxic effect as the free CPT.

The cytotoxic effect of the peptide CPT conjugate was not tested on HEK normal cells, as the peptide did not bind and internalized to EGFR−ve.

Table 4 presents the cytotoxicity assay results as IC₅₀ values (μM) of P6, P6-CPT, P9, P9-CPT on H1299, DKMG cells, wherein values were calculated using non-linear regression.

TABLE 4

| | H1299 | | | | | DKMG | | |
|---|---|---|---|---|---|---|---|---|
| Incubation | P6 | P6-CPT | P9 | P9-CPT | CPT | P9 | P9-CPT | CPT |
| 6 w 24 h | >50% | >50% | >50% | >50% | >50% | >50% | >50% | >50% |
| 6 w 48 h | >50% | 32± | >50% | 21.5 ± 2.1 | 6.7 ± 1.8 | >50% | 32 ± 1.6 | 8.5 ± 1.0 |
| 24 h | 27.5 ± 0.8 | 14.5± | 22.3 ± 0.2 | 8.4 ± 0.5 | 54.8 ± 2 | 16.4 ± 1.9 | 6.76 ± 1.4 | 25 ± 1.3 |
| 48 h | 32± | 5.5± | 27.3 ± 1.7 | 2.2 ± 0.3 | <45% | 17.5 ± 2.3 | 2.7 ± 1.0 | <50% |
| 72 h | 28.8± | <27% | 22.5 ± 0.0 | 1.2 ± 0.1 | <15% | 17.5 ± 1.6 | 0.2 ± 0.1 | <40% |

### In Vivo Targeting:

FITC-labeled P6 or P11 peptides were injected intravenously into mice bearing H1299 and K562. Signal from the targeted tumor compared to other tissues was determined for each animal.

FITC-labeled P6 or P11 accumulates in the H1299 tumor compared to the K562 tumor; indicating that the peptides discriminate between the EGFR positive tumor and EGFR negative tumor in vivo. No significant accumulation of the P6 and P11 peptide was observed in other organs. One metastasis was found in the liver which was also positive for FITC.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1-2. (canceled)

3. A cyclic peptide, having a sequence selected from the group consisting of:
(SEQ ID No. 1)
CLRWRFGRC;
(SEQ ID No. 2)
CSAETVESC;
(SEQ ID No. 3)
CVRWRFGRC;
(SEQ ID No. 4)
CLAVEVRPC;
(SEQ ID No. 5)
CPNDSYHQC;
(SEQ ID No. 6)
CHVPGSYIC;
(SEQ ID No. 7)
CWHSLSLAC;
(SEQ ID No. 8)
CSALWASHC;
(SEQ ID No. 9)
CVNAMQSYC;
(SEQ ID No. 10)
CNWLSRTEC;
and
(SEQ ID No. 11)
CAQYTPGRC,
and any C-terminus amide, salt, hydrate or solvate thereof.

4. The cyclic peptide of claim 3, further comprising a moiety of a bioactive agent, attached thereto.

5. The cyclic peptide of claim 4, wherein said bioactive agent is selected from the group consisting of a drug, cytotoxic agent, an imaging agent, a diagnostic agent and a labeling agent.

6. The cyclic peptide of claim 3, for use in targeted drug delivery to cells overexpressing the EGFR and EGFRvIII mutation.

7. A conjugate comprising a moiety of the cyclic peptide of claim 3, and a moiety of a bioactive agent.

8. The conjugate of claim 7, wherein said moiety of the cyclic peptide and said moiety of said bioactive agent are connected via a linking moiety, said linking moiety is biodegradable.

9. (canceled)

10. The conjugate of claim 8, wherein said linking moiety comprises a spacer moiety.

11. The conjugate of claim 8, wherein said linking moiety is selected from the group consisting of a γ-aminobutyric acid (GABA) moiety, a glutathione moiety, a lysine moiety, a succinic acid moiety, a 2-amino-5-(carbamoylamino) pentanoic acid (PABA) moiety, a citrulline moiety, a valine-citrulline-PABA moiety, and any combination thereof.

12. The conjugate of claim 7, wherein said bioactive agent is a cytotoxic agent.

13. The conjugate of claim 12, wherein said cytotoxic agent is selected from the group consisting of camptothecin (CTP), doxorubicin (DOX), monomethyl auristatin F (MMFA), and 7-ethyl-10-hydroxycamptothecin (SN38).

14. The conjugate of claim 12, exhibiting higher cytotoxicity to cells overexpressing the EGFR and EGFRvIII mutation than the free cytotoxic agent.

15. A pharmaceutical composition comprising the conjugate of claim 7, and a pharmaceutically acceptable carrier, diluent, or excipient.

16. The pharmaceutical composition of claim 15, being packaged in a packaging material and identified in print, in or on said packaging material, for use in the treatment of a medical condition.

17.-19. (canceled)

20. The pharmaceutical composition of claim 16, wherein said medical condition is treatable by said bioactive agent.

21. The pharmaceutical composition of claim 16, wherein said medical condition is associated with cells overexpressing the EGFR and EGFRvIII mutation.

22. The pharmaceutical composition of claim 16, wherein said bioactive agent is a cytotoxic agent.

23. The pharmaceutical composition of claim 22, wherein said medical condition is cancer.

24. A method for treating a medical condition associated with cells overexpressing the EGFR and EGFRvIII mutation, comprising administering to a subject in need thereof a therapeutically effective amount of the conjugate of claim 7.

25. The method of claim 24, wherein said medical condition is cancer.

26. A method for diagnosing a disease associated with cells overexpressing the EGFR and EGFRVIII mutation, comprising using the conjugate of claim 7 as a diagnostic agent in imaging or detection techniques.


SEQUENCE LISTING (11 sequences)

SEQ ID NO: 1 - protein, 9 aa, synthetic construct
  DISULFID 1..9: note = disulfide-looped via two terminal cysteines
  MOD_RES 9: note = C-terminus may be amidated
CLRWRFGRC

SEQ ID NO: 2 - protein, 9 aa, synthetic construct
  DISULFID 1..9: note = disulfide-looped via two terminal cysteines
  MOD_RES 9: note = C-terminus may be amidated
CSAETVESC

SEQ ID NO: 3 - protein, 9 aa, synthetic construct
  DISULFID 1..9: note = disulfide-looped via two terminal cysteines
  MOD_RES 9: note = C-terminus may be amidated
CVRWRFGRC

SEQ ID NO: 4 - protein, 9 aa, synthetic construct
  DISULFID 1..9: note = disulfide-looped via two terminal cysteines
  MOD_RES 9: note = C-terminus may be amidated
CLAVEVRPC

SEQ ID NO: 5 - protein, 9 aa, synthetic construct
  DISULFID 1..9: note = disulfide-looped via two terminal cysteines
  MOD_RES 9: note = C-terminus may be amidated
CPNDSYHQC

SEQ ID NO: 6 - protein, 9 aa, synthetic construct
  DISULFID 1..9: note = disulfide-looped via two terminal cysteines
  MOD_RES 9: note = C-terminus may be amidated
CHVPGSYIC

SEQ ID NO: 7 - protein, 9 aa, synthetic construct
  DISULFID 1..9: note = disulfide-looped via two terminal cysteines
  MOD_RES 9: note = C-terminus may be amidated
CWHSLSLAC

SEQ ID NO: 8 - protein, 9 aa, synthetic construct
  DISULFID 1..9: note = disulfide-looped via two terminal cysteines
  MOD_RES 9: note = C-terminus may be amidated
CSALWASHC

SEQ ID NO: 9 - protein, 9 aa, synthetic construct
  DISULFID 1..9: note = disulfide-looped via two terminal cysteines
  MOD_RES 9: note = C-terminus may be amidated
CVNAMQSYC

SEQ ID NO: 10 - protein, 9 aa, synthetic construct
  DISULFID 1..9: note = disulfide-looped via two terminal cysteines
  MOD_RES 9: note = C-terminus may be amidated
CNWLSRTEC

SEQ ID NO: 11 - protein, 9 aa, synthetic construct
  DISULFID 1..9: note = disulfide-looped via two terminal cysteines
  MOD_RES 9: note = C-terminus may be amidated
CAQYTPGRC